(19) United States
(12) Patent Application Publication
(10) Pub. No.: US 2026/0132103 A1
(43) Pub. Date: May 14, 2026

(54) Bumetanide Derivatives for the Therapy of Stroke and Other Neurological Diseases/Disorders Involving NKCCs

(71) Applicants: University of Pittsburgh - Of the Commonwealth System of Higher Education, Pittsburgh, PA (US); The United States Government as represented by the Department of Veterans Affairs, Washington, DC (US); Universität Wien, Wien (AT)

(72) Inventors: Thomas Erker, Vienna (AT); Philipp Schreppel, Vienna (AT); Dandan Sun, Pittsburgh, PA (US)

(21) Appl. No.: 19/035,380

(22) Filed: Jan. 23, 2025

Related U.S. Application Data
(62) Division of application No. 17/045,395, now abandoned.
(60) Provisional application No. 62/653,708, filed on Apr. 6, 2018.

(51) Int. Cl. C07C 311/39; A61P 9/10; A61P 35/00

(52) U.S. Cl. CPC C07C 311/39; A61P 9/10; A61P 35/00

(57) ABSTRACT

The present invention relates to bumetanide derivatives of formula (I) as well as pharmaceutical compositions comprising these compounds for use in the treatment or prevention of neurological diseases/disorders involving Na⁺—K⁺-2Cl⁻—cotransporters (NKCCs), such as stroke, traumatic brain injury (TBI), spinal cord injury (SCI), peripheral nerve injury (PNI), brain edema, or glioma, and particularly for use in the treatment or prevention of stroke. The invention likewise relates to a method of treating or preventing a neurological disease or disorder involving an NKCC, such as stroke, TBI, SCI, PNI, brain edema, or glioma, the method comprising administering a compound of formula (I) to a subject in need thereof.

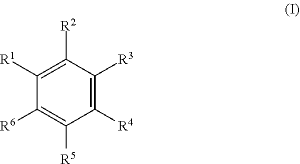

### CROSS-REFERENCE TO RELATED APPLICATIONS

This application is a divisional application of U.S. patent application Ser. No. 17/045,395, filed Apr. 5, 2019, which is the United States national phase of International Application No. PCT/EP2019/058656 filed Apr. 5, 2019, which claims priority to U.S. Provisional Application No. 62/653,708 filed Apr. 6, 2018, the disclosures of which are hereby incorporated herein by reference in their entirety.

### STATEMENT REGARDING FEDERAL FUNDING

This invention was made with government support under Grant No. NS038118 awarded by the National Institute of Health. The government has certain rights in the invention.

### BACKGROUND OF THE INVENTION

The present invention relates to bumetanide derivatives of formula (I) as well as pharmaceutical compositions comprising these compounds for use in the treatment or prevention of neurological diseases/disorders involving Na⁺—K⁺-2Cl⁻—cotransporters (NKCCs), such as stroke, traumatic brain injury (TBI), spinal cord injury (SCI), peripheral nerve injury (PNI), brain edema, or glioma, and particularly for use in the treatment or prevention of stroke. The invention likewise relates to a method of treating or preventing a neurological disease or disorder involving an NKCC, such as stroke, TBI, SCI, PNI, brain edema, or glioma, the method comprising administering a compound of formula (I) to a subject in need thereof.

Na⁺—K⁺—Cl⁻—Co-Transporters

The intracellular Cl⁻ concentration ([Cl⁻]ᵢ) is mostly controlled by the chloride-cation-cotransporters (CCCs) of the SLC12 gene family (Maa et al., 2011). These transporters are among the most important ion transporters in multicellular organisms and are crucial for survival (Alessi et al., 2014). Na⁺—K⁺-2Cl⁻—cotransporters (NKCCs) transfer Cl⁻ into the cell and K⁺—Cl⁻—cotransporters (KCCs) are outwardly directed (Munoz, DeFelipe, & Alvarez-Leefmans, 2007). These CCCs are intrinsic membrane proteins that use the energetically favorable transmembrane gradients of potassium and sodium ions to transport Cl⁻ across membranes. These gradients are established by active primary transport of the ouabain-sensitive Na⁺—K⁺—ATPase (Alessi et al., 2014). The Cl⁻ transport is performed electroneutrally therefore without any net charge movement across the membrane (Payne et al., 2003). NKCC1 and NKCC2 use the inward sodium current to transport Cl⁻ into the cell above its equilibrium level. KCC1, KCC2, KCC3 and KCC4 use the potassium gradient to transport Cl⁻ out of the cell, lowering [Cl⁻]ᵢ below the equilibrium level (Maa et al., 2011). NKCC1 is widely distributed throughout the body and expressed in neurons, glial cells, the choroid plexus and vascular endothelium, whereas NKCC2 is primarily expressed in the kidney (Maa et al., 2011). NKCC1 and NKCC2 share 60% homology at the protein level (Markadieu, N., Delpire, E., 2014). The cellular Cl⁻ efflux and influx is also regulated by two serine-threonine kinases SPAK and OSR1 that phosphorylate critical N- and C-residues of NKCCs and KCCs. Thus, they activate NKCCs and cause Cl⁻ influx, but at the same time they inhibit KCCs and Cl⁻ efflux (Alessi et al., 2014). In the mammalian central nervous system (CNS) the [Cl⁻]ᵢ determines the strength and direction of GABAergic neurotransmission (Kahle & Staley, 2008). In the adult CNS there are very low levels of [Cl⁻]ᵢ and the activation of the GABA_{A} receptor leads to an influx of Cl⁻ into the cell, causing hyperpolarization and inhibition (Khanna, Walcott, & Kahle, 2013). The immature brain of neonates, on the other hand, exhibits a much higher [Cl⁻]ᵢ, so that activating the GABA_{A} receptor causes an efflux of Cl⁻, which depolarizes the neuron and leads to synaptic excitation (Kahle & Staley, 2008).

### NKCC2

NKCC2 is expressed at the apical membrane of the epithelial cells in the ascending limb of henle, which reabsorbs around 20-30% of the NaCl filtered by the glomerulus (Ares G., Caceres P., Ortiz P., 2011). The main function of the ascending limb of henle is the reabsorption of NaCl, but no water. This leads to a further dilution of the forming urine in the tubule lumen. NKCC2 is also expressed in the Macula densa. The Macula densa cells act as NaCl-sensors and are able to adjust the glomerular filtration by either vasoconstriction or vasodilation of the afferent arteriole. A decrease of the tubular NaCl concentration will lead to a vasodilation of the afferent arteriole and a release of renin by the granular cells. In contrast, an increase of the tubular NaCl concentration will lead to a vasoconstriction of the afferent arteriole and thereby a decrease of glomerular filtration. This mechanism is known as the tubuloglomerular feedback and NKCC2 has been shown to play a very important role in sensing high NaCl concentration (Peti-Peterdi, J., Harris, R., 2010).

### NKCC1

In contrast to NKCC2, NKCC1 is widely distributed throughout the body and has a lot of different functions. It is highly expressed in the inner ear spiral and vestibular ganglia. Regulation of [Cl⁻]ᵢ seems to be an important function of NKCC1 in adult neurons, that are not located in the CNS. In the immature brain, however, there is an increased expression of NKCC1 and these elevated levels of [Cl⁻]ᵢ seem to have developmental effects (Dzhala et al., 2005). NKCC1 is also highly expressed in the salivary gland, where it participates in the secretion of fluid and mucine. It is also expressed in the intestine, where it is also involved in fluid excretion. The most striking deficit of NKCC1 knockout mice is deafness and imbalance, which originates from the fact that NKCC1 is highly expressed in the inner ear. It plays a major role in afferent neurons. In the CNS it is only elevated in immature neurons and plays an important role in neuronal maturation. NKCC1 knockout mice also suffer from hypotension and male infertility. The hypotension originates from a decreased vascular tone (Markadieu, N., Delpire, E., 2014).

### GABA

Gamma-Aminobutyric acid (GABA) is the primary inhibitory neurotransmitter in the adult mammalian brain (Dzhala et al., 2005). However, GABA-mediated signaling also plays a key role in all important developmental steps such as cell proliferation (Owens & Kriegstein, 2002). The GABA_{A} receptor is a ligand-gated chloride channel that is opened by GABA docking to its binding-site. The receptor performs a conformational change, which allows Cl⁻ to passively flow either into the cell or out of the cell, depending on the chloride equilibrium concentration (Maa et al., 2011). The channel also allows bicarbonate to permeate the channel pore, but less efficiently than chloride (Owens & Kriegstein, 2002). Inflow of Cl⁻ causes hyperpolarization, outflow of Cl⁻ on the other hand, results in depolarization. In the immature brain GABA has a depolarizing effect, it excites neurons and can therefore cause seizures (Dzhala et al., 2005). The depolarizing effect of GABA is crucial for brain development. It has been shown that the GABA_{A} receptor influences DNA-synthesis, proliferation and neuronal migration (Owens & Kriegstein, 2002). In the immature neuron there is a delicate equilibrium between inhibition and excitation. This balance plays an important role during the early stages of brain development. Excessive inhibition leads to failure in neuronal growth and synaptic maturation, whereas excessive excitation can cause seizures and even excitotoxic death (Maa et al., 2011).

Recovery after Stroke

Stroke is the fifth leading cause of death in the US and 795000 people in the US have a stroke each year. A block or rupture of a blood vessel supplying the brain with blood is causing a stroke. Stroke is also a major cause of disability, it reduces mobility in more than half of stroke survivors older than 65 (Centers for Disease Control and Prevention, (2016) Stroke Fact Sheet). Ischemic stroke promotes adult neurogenesis, but it seems that these new neurons have rather limited capabilities to survive in the long term. An ischemic stroke can cause an imbalance in the expression of NKCC and KCC, leading to an increased [Cl⁻]ᵢ and ultimately to a shift in GABAergic activation from hyperpolarizing to depolarizing. Chronic post-treatment with bumetanide can enhance the migration of neuroblasts towards the damaged striatum and can also enhance the survival of these new-born neurons. Furthermore, the behavioral assessment showed improved beam-walking performance. Therefore, bumetanide and its derivatives might cause a favorable microenvironment for newborn neurons that enhances their generation and survival (Xu W., et al. 2016). Thus, the derivatives can be used to enhance regeneration and reduce damage after ischemic stroke, but also in other diseases (e.g., Alzheimer's) for memory enhancement.

### Spinal Cord Injury

Between 250000 and 500000 people worldwide suffer spinal cord injury (SCI) each year. 90% of the causes for SCI are traumatic causes such as road traffic crashes, falls or violence. People affected by SCI have a two to five times increased risk to die prematurely, have lower school enrollment and economic participation. Symptoms of SCI depend on the severity of injury and its location, but most patients experience chronic pain (WHO (2013) Spinal Cord Injury Fact Sheet No 384). Injury and noxious input can lead to a long-lasting increase in spinal cord neural excitability that may cause chronic pain. Spinal cord injury (SCI) can transform the activation of GABA-channels from hyperpolarizing to depolarizing. The effect of SCI was linked to a downregulation of KCC2 leading to a high [Cl⁻]ᵢ leading to the shift of GABAergic activation. Neural injury seems to push the spinal systems towards a state of early development, where GABA has a depolarizing effect. This depolarizing effect might cause the development of chronic pain and spasticity. Bumetanide can restore normal GABAergic function by blocking the NKCC and restoring normal [Cl⁻]ᵢ concentration (Huang Y., et al. 2016). There is a big need of an improved therapy for those patients suffering from chronic pain caused by SCI.

### Brain Edema

Cerebral edema (brain edema) causes intracranial hypertension (ICH) which leads to severe outcome of patients in the clinical setting. Effective anti-edema therapy may significantly decrease the mortality in a variety of neurological conditions. At present drug treatment is a cornerstone in the management of cerebral edema. Osmotherapy has been the mainstay of pharmacological therapy. Mannitol and hypertonic saline (HS) are the most commonly used osmotic agents. The inhibitors of Na/H exchanger, NKCC attenuate brain edema formation through inhibition of excessive transportation of ion and water from blood into the cerebral tissue (Deng Y., et al. 2016). NKCC inhibitors can thus be used in the therapy of cerebral edema.

### Autism Spectrum Disorder

Autism spectrum disorder (ASD) is a range of complex neurodevelopmental disorders that are characterized by repetitive and characteristic patterns of behavior and difficulties of social interaction and communication. ASD consists of a range of different disorders, with autism being the most severe form. Other forms are Asperger syndrome, childhood disintegrative disorder and pervasive developmental disorders as part of ASD. One in 68 children is affected by a ASD and boys are significantly more likely than girls to develop ASD. It occurs throughout all racial and ethnics groups and across all socioeconomic levels (NINDS Autism Spectrum Disorder Fact Sheet 2016). Delivery in rodents plays a very important role, causing neuroprotective and analgesic effects in newborns. These effects are caused by an oxytocin-mediated decrease of [Cl⁻]ᵢ. In two models of autism in rodents (VPA rats and FRX mice) this sequence is abolished in CA3 pyramidal neurons. It can be restored by administering bumetanide, which leads to a restoration of the GABA developmental sequence and those rats do not show any autistic phenotype in rodent offspring (Tyzio R. et al. 2014). Bumetanide has also been used in an open label trial with seven patients diagnosed with autism. The patients were treated with bumetanide for 10 months and bumetanide caused an improvement in emotion recognition and enhanced the activation of brain regions involved in social and emotional perception (Hadjikhani N., et al. 2015). Since there is no cure and only very limited treatment options available for ASD, there is a strong need for improved therapy.

### Schizophrenia

Schizophrenia is characterized by distortions in emotions, perceptions, thinking, behavior, sense of self and language. Many patients experience hearing voices and delusions. More than 21 million people are affected worldwide and it is associated with considerable disability. Patients are 2-2.5 times more likely to die prematurely and discrimination and stigma are very common (WHO (2016) Schizophrenia Fact Sheet). The neurophysiological basis of schizophrenia remains poorly understood, but many studies suggest that a dysregulation of cortical GABA transmission might be the cause of schizophrenia. In a recent study a gain-of-function missense variant in SLC12A2, encoding the bumetanide sensitive NKCC1 cotransporter, was identified in human schizophrenia. Functional experiments showed that this variant of NKCC1 is a gain-of-function variant, increasing Cl⁻-dependent activity even in conditions in which the transporter is normally functionally silent (hypotonicity) (Merner, N. D., et al. 2016). Another study found a KCC loss-of-function variant in human schizophrenia (Merner, N. D., et al. 2015). In both cases (gain-of-function of NKCC, loss-of-function of KCC) the [Cl⁻]ᵢ is increased, which might lead to a disruption of GABA neurotransmission. Blocking the NKCC would lead to a normalization of [Cl⁻]ᵢ and reverse GABA signaling back to hyperpolarizing.

### Down Syndrome

Down syndrome is the most frequent genetic cause that leads to intellectual disability.

Adults and children, who suffer from Down syndrome express lower than normal intelligence quotients, learning deficits and memory impairment. It is caused by extra genetic material in chromosome 21. This can be due to a process called nondisjunction, where the genetic material fails to separate resulting in an extra chromosome (trisomy 21). The prevalence is around 1 to 1000 births worldwide, which means that each year around 3000 to 5000 children are born with this disorder (WHO (2017), Genes and chromosomal diseases). Altered GABAergic transmission contributes considerably to the learning and memory deficits in mouse models. A recent publication has shown that bumetanide was able to restore normal GABAergic transmission and reduce cognitive impairments (Deidda, G., et al. 2015). Based on this study, bumetanide and derivatives thereof are a promising approach for the treatment of mental disability in patients with Down syndrome.

### Glioma

As described above, NKCC1 is an active cotransporter that brings Na⁺, K⁺, and 2Cl⁻ into the cell and plays an important role in intracellular Cl⁻ accumulation (Cuddapah and Sontheimer 2011; Garzon-Muvdi et al. 2012) which lead to cell volume regulation and migration through the electrochemical driving force for Cl⁻ efflux to osmotically release cytoplasmic water (Cuddapah and Sontheimer 2011). NKCC1 significantly displays higher expression levels in human glioma cells than normal control adult cortex (Aronica et al. 2007) and localizes to the leading edge of human glioma cells (Haas and Sontheimer 2010). It has been reported that NKCC1 protein serves as a protein scaffold to cofilin and facilitates its localization at the plasma membrane and regulates the actin cytoskeleton in primary glioblastoma (GBM) cells (Schiapparelli et al. 2017). NKCC1 also promotes epithelial-mesenchymal transition-like process via facilitating the binding of Rac1 and RhoA to GTP during glioma cell invasion in GBM animal model (Ma et al. 2019). Pharmacological inhibition of NKCC1 and shRNA-knockdowns of NKCC1 do not affect cell motility but manifest only when cells had to undergo volume changes during migration (Haas and Sontheimer 2010). More interestingly, elevated intracellular Cl⁻ concentration ([Cl⁻]ᵢ) was also found in peritumoral neurons and led to an efflux of Cl⁻ that causes a characteristic depolarizing response to γ-aminobutyric acid (GABA)-mediated chloride channel opening (Habela et al. 2009). Compromised GABAergic inhibition contributes to tumor-associated epilepsy (Campbell et al. 2015). Blockade of NKCC1 to offset the loss of KCC2 reduced the seizure susceptibility in glioma-implanted mice (MacKenzie et al. 2016). It was recently reported that NKCC1 was further triggered in TMZ-mediated apoptosis by replenishing K⁺ᵢ and Cl⁻ᵢ (Algharabil et al. 2012) and was detected by immunoblotting in glioma cells in vitro and immunofluorescence staining in two intracranial syngeneic mouse glioma models in vivo. Blocking NKCC1 protein function represents an attractive approach to potentiate TMZ-induced cytotoxicity and improve the outcome of glioma patients.

### Bumetanide and its Derivatives

Bumetanide, i.e. 3-(butylamino)-4-phenoxy-5-sulfamoylbenzoic acid, is a loop diuretic (high ceiling diuretic) with a well-established clinical profile (Younus and Reddy 2018), which blocks both NKCC1 and NKCC2 and has been approved by the FDA and the EMA for the treatment of edema, particularly edema associated with congestive heart failure, hepatic disease and renal disease, including the nephrotic syndrome; bumetanide and/or certain derivatives thereof have also been proposed for various other therapeutic applications (Lykke K et al., Br J Pharmacol. 2015,172(18): 4469-80; Töllner K et al., Eur J Pharmacol. 2015, 746:78-88; Töllner K et al., Ann Neurol. 2014, 75(4):550-62; Erker T et al., Epilepsia. 2016, 57(5):698-705; Louie J C et al., Physiol Rep. 2016, 4(22). pii: e13024). However, due to its very polar carboxylic acid group, bumetanide can barely penetrate through cell membranes, which severely limits the therapeutic potential of this drug. In particular, as bumetanide is tightly bound to plasma proteins and nearly completely ionized at physiologic pH, its ability to cross the blood-brain barrier is hampered (\ddot{T}ollner et al. 2014). The major adverse effects of bumetanide stem from its diuretic action at the level of the nephron NKCC, leading to excessive fluid loss, electrolyte depletion, hypokalemia, dehydration, hypotension, and possibility of thrombus and emboli (Younus and Reddy 2018). Furthermore, bumetanide has been reported to lead to severe hearing impairment in patients after the application of high doses (Allegaert et al. 2016). In view of the weak therapeutic activity, the undesirably strong diuretic effect and the related adverse effects of bumetanide, there is thus an urgent and unmet need for novel and/or improved therapeutic agents that can be used for the therapy of NKCC-implicated disorders and do not suffer from the disadvantages associated with bumetanide.

Certain other (hetero)arylsulfonamides, benzoic acids, thiophenes, pyrazolopyrimidines or other derivatives are described, e.g., in Feit P W et al., J Med Chem. 1976, 19(3):402-6; Feit P W et al., J Med Chem. 1977, 20(12):1687-91; Consiglio G et al., ARKIVOC. 2002, 11:104-17; Hauck S et al., Bioorg Med Chem. 2016, 24(22):5717-29; Moni L et al., Synthesis. 2016, 48(23):4050-9; Moni L et al., Molecules. 2016, 21(9):1153/1-1153/9; Palfrey H C et al., American Journal of Physiology. 1984, 246(3, Pt. 1):C242-C246; Englert H et al., Archiv der Pharmazie (Weinheim, Germany). 1983, 316(5):460-3; Nielsen O T et al., Am Chem Soc Symp Ser, Diuretic Agents. 1978, 83:12-23; Petzinger E et al., Am J Physiol. 1993, 265(5 Pt 1):G942-54; AU-A-521892; CN-A-104926804, DE-A-1966878; DE-A-2654795; GB-A-1523632; U.S. Pat. Nos. 3,985,777; 4,010,273; US 2014/066504; WO 2008/052190; WO 2010/083442; WO 2010/085352; WO 2012/018635; WO 2013/087090; WO 2014/157635; WO 2014/196793; and WO 2014/039454.

It is thus an object of the present invention to provide novel and/or improved active agents for the therapy of neurological diseases/disorders involving NKCCs, particularly NKCC1, such as stroke, traumatic brain injury, spinal cord injury, peripheral nerve injury, brain edema, or glioma.

### SUMMARY OF THE INVENTION

In the context of the present invention, it has been found that the bumetanide derivatives of formula (I) as described and defined herein can be used as inhibitors of Na⁺—K⁺-2Cl⁻—cotransporters (NKCCs), particularly as NKCC1 inhibitors. Moreover, it has been found that the bumetanide derivatives of formula (I) exhibit considerably improved properties, particularly with respect to penetration, diuresis and metabolic stability. The compounds provided herein thus show an increased lipophilicity and improved skin penetration, a significantly reduced diuretic activity, an improved metabolic stability and, overall, an enhanced therapeutic effectiveness. This makes the compounds according to the invention highly advantageous for therapeutic applications, including for the treatment or prevention of neurological diseases or disorders involving NKCCs, particularly NKCC1.

The compounds of formula (I) according to the present invention can hence advantageously be used for the treatment or prevention of neurological diseases/disorders involving NKCCs, including any of the diseases/disorders described herein above in the background of the invention section. The present invention also provides compounds of formula (I) that are selective inhibitors of NKCC1, particularly compounds that inhibit NKCC1 more potently than NKCC2, which renders these compounds especially suitable for the treatment or prevention of NKCC1-implicated diseases/disorders as well as for therapeutic applications in which a high-ceiling diuretic effect is undesirable. Some of the compounds of the invention furthermore show an advantageous water-solubility. The invention also provides compounds of formula (I) that comprise a carboxylic ester group and can be hydrolyzed by esterases in the skin of a patient to release more polar therapeutically active compounds which, due to their increased polarity, will not easily be transported back to the skin surface and will thus accumulate at the desired target site (“metabolic trapping”), resulting in a more pronounced and/or prolonged therapeutic effect.

All these properties render the compounds of the present invention highly suitable as medicaments for inhibiting NKCCs, particularly NKCC1, and thus for the therapeutic intervention in neurological diseases/disorders involving NKCCs and, in particular, for the treatment or prevention of stroke, traumatic brain injury, spinal cord injury, peripheral nerve injury, brain edema, or glioma.

Furthermore, as demonstrated in a mouse model of ischemic stroke, it has been found that the compounds of formula (I) according to the invention exhibit a particularly advantageous therapeutic efficacy in the treatment of stroke. Thus, as described in Example 3, treatment with an exemplary compound of formula (I) resulted in an efficient reduction of brain infarction and cerebral edema after stroke, as well as a considerably improved survival rate after ischemic stroke, particularly in comparison to the known drug bumetanide and a known prodrug of bumetanide. Moreover, the exemplary compound of formula (I) was found to improve neurological functions and sensorimotor deficits after ischemic stroke faster and more effectively than bumetanide, and to exhibit improved neuroprotective effects. This compound was furthermore found to be highly effective in reducing ischemic damage in a mouse model of stroke with hypertension comorbidity (pdMCAO stroke model). These findings show that the compounds of formula (I) are highly advantageous for the treatment of neurological diseases/disorders involving NKCCs, particularly for the therapy of stroke. It has further been demonstrated that the compounds of formula (I) are effective in the therapy of glioma, as described in detail in Example 4.

Accordingly, the present invention provides a compound of the following formula (I) or a pharmaceutically acceptable salt or solvate thereof

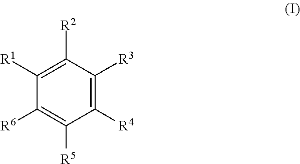

for use in the treatment or prevention of a neurological disease or disorder involving an NKCC (particularly NKCC1), such as, e.g., stroke, traumatic brain injury, spinal cord injury, peripheral nerve injury, brain edema, or glioma.

In formula (I), R¹ is selected from —(C₁₋₄ alkylene)-NH—(C₁₋₄ alkylene)-R¹¹, —COO—(C₁₋₄ alkylene)-R¹¹, —O—CO—(C₁₋₄ alkylene)-R¹¹, —CO—(C₁₋₄ alkylene)-R¹¹, —CO—NH—(C₁₋₄ alkylene)-R¹¹, —CO—N(C₁₋₄ alkyl)-(C₁₋₄ alkylene)-R¹¹, —NH—CO—(C₁₋₄ alkylene)-R¹¹ and —N(C₁₋₄ alkyl)-CO—(C₁₋₄ alkylene)-R¹¹.

R¹¹ is independently selected from —CF₃, —CN and halogen.

R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —S H, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl).

R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl).

R⁴ is selected from —O—R⁴¹, —S—R⁴¹, —NH—R⁴¹, —N(C₁₋₆ alkyl)-R⁴¹, halogen, hydrogen, carbocyclyl and heterocyclyl, wherein said carbocyclyl and said heterocyclyl are each optionally substituted with one or more groups R⁴².

R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more groups R⁴³ Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl).

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl).

R⁵ is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —S H, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl).

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl).

The present invention also relates to a pharmaceutical composition comprising a compound of formula (I) or a pharmaceutically acceptable salt or solvate thereof, in combination with a pharmaceutically acceptable excipient, for use in the treatment or prevention of a neurological disease or disorder involving an NKCC (particularly NKCC1), such as, e.g., stroke, traumatic brain injury, spinal cord injury, peripheral nerve injury, brain edema, or glioma.

Moreover, the present invention relates to the use of a compound of formula (I) or a pharmaceutically acceptable salt or solvate thereof in the preparation of a medicament for the treatment or prevention of a neurological disease or disorder involving an NKCC (particularly NKCC1), such as, e.g., stroke, traumatic brain injury, spinal cord injury, peripheral nerve injury, brain edema, or glioma.

The invention likewise relates to a method of treating or preventing a neurological disease or disorder involving an NKCC (particularly NKCC1), the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. The neurological disease or disorder may be, e.g., stroke, traumatic brain injury, spinal cord injury, peripheral nerve injury, brain edema, or glioma. It will be understood that a therapeutically effective amount of the compound of formula (I) or the pharmaceutically acceptable salt or solvate thereof, or of the pharmaceutical composition, is to be administered in accordance with this method.

### BRIEF DESCRIPTION OF THE FIGURES

FIGS. 1A-1C: Efficacy of NKCC1 inhibitors in reducing ischemic infarction, cerebral edema, and the post-stroke survival in normotensive mice after tMCAO (see Example 3). (1A) Experimental protocol and data collection. Transient middle cerebral artery occlusion (tMCAO) was induced in normal C57/b6 mice. DMSO, BMT (10 mg/kg body weight), STS5 (13 mg/kg) or STS66 (12 mg/kg) was administered via intraperitoneal injection (i.p.) as described in method. (1 B) Representative TTC staining images in normotensive mice at 24 h reperfusion with quantitative analysis of infarct volume. Data are mean±SD, n=6-13 (male, n=3-9; female, n=3-4, *p<0.05). Scale: 5 mm. (1C) Percentage hemisphere swelling at 24 h reperfusion. Data are mean±SD, n=6-13, *p<0.05. (1D) Body weight change during 1-14 days after tMCAO. Data are mean±SD, n=5-9 (male, n=4; female, n=2, *p<0.05). (1E) Survival curve during 1-14 day after tMCAO. Data are mean±SD, n=5-9 (male, n=3-7; female, n=2, *p<0.05).

FIGS. 2A-2D: Efficacy of NKCC1 inhibitors on improving neurological function in mice after tMCAO (see Example 3). (A) Neurological score, (B) corner test, (C) foot-fault test, and (D)-(E) adhesive tape test during 1-14 day after tMCAO. Data are mean±SD, n=4-9. *p<0.05 for DMSO vs STS66, **p<0.05 for DMSO vs STS5, ***p<0.05 for DMSO vs BMT, #p<0.05 for BMT vs STS66, ###p<0.05 for STS5 vs STS66.

FIG. 3: Effects of NKCC1 inhibitors against the worsened ischemic stroke in AngII-induced hypertensive mice after pdMCAO (see Example 3). (A) Experimental protocol in AngII-induced hypertensive C57/B6 mice. Permanent distal middle cerebral artery occlusion (pdMCAO) was induced in mice at day 14 post-AngII infusion. DMSO, BMT or STS66 was administered in mice via i.p. with the initial half dose at 3 h and the second half dose at 8 h after pdMCAO. (B) Representative TTC-stained mouse coronal brain sections at 2-8 mm posterior of the frontal pole were shown. TTC staining was performed at 24 h post pdMCAO. Scale: 5 mm. (C) Brain infarct volume was calculated. Data are mean±SD. Saline (n=4), AngII (n=5); *p<0.05 vs. saline control.

FIGS. 4A-4B: Rb⁺ influx was measured in GL26 and SB28-GFP cells in either isotonic (10 min) or hypertonic (5 min) solutions using ICR 8000 machine. (4A) Cells were exposed to isotonic or hypertonic solutions with BMT (10 μM) or STS66 (10 μM). (4B) Cells were exposed to isotonic or hypertonic solutions with different concentrations of STS66 (0 to 40 μM). Results are expressed as means±SEM from four independent experiments. **P<0.01, ***P<0.001, ****P<0.0001.

FIG. 5: GL26 cells and SB28-GFP cells were first exposed to BMT (10 μM) and different concentration of STS66 (10, 20 and 40 μM) for 48 h, and Rb⁺ influx was measured in either isotonic (10 min) or hypertonic (5 min) solutions using ICR 8000 machine. Results are expressed as means±SEM from four independent experiments. *P<0.05, **P<0.01, ***P<0.001, ****P<0.0001.

FIGS. 6A-6B: GL26 and SB28-GFP cells were exposed to BMT (10 μM), STS66 (10 μM), TMZ (100 μM) or combined for 6 h and cell lysates were harvested for immunoblotting of pNKCC1 and tNKCC1 proteins. Data are means±SEM from six independent experiments. *P<0.05, **P<0.01, ***P<0.001.

FIG. 7: BrdU incorporation of GL26 cells and SB28-GFP cells following 48 h drug treatment. Data are means±SEM from three independent experiments. **P<0.01, ***p<0.001, ****p<0.0001.

FIG. 8: The combinatorial regimen of BMT, STS66, and TMZ may increase glioma bearing mouse survival. (A) Experimental protocol and location of data collection. GL26 and SB28-GFP glioma cells were injected into the right striatum of C57BL6/J mice. Starting 7 d.p.i., mice received either vehicle PBS-DMSO (10 ml/kg/day, i.p.), TMZ (10 mg/kg/day, i.p.), BMT (5 mg/kg, twice a day, i.p.), STS66 (6 mg/kg, twice a day, i.p.) and combination treatment for T+B (10+5 mg/kg/twice a day, i.p.) and T+S (10+6 mg/kg/twice a day, i.p.) for 5 consecutive days. (B) Kaplan-Meier survival curve of GL26 (n=5) and (C) SB28-GFP tumor-bearing mice (n=3).

### DETAILED DESCRIPTION OF THE INVENTION

As described above, the present invention relates to the treatment or prevention of a neurological disease or disorder involving (or mediated by) an NKCC, particularly a neurological disease or disorder involving (or mediated by) NKCC1, such as, e.g., stroke (particularly ischemic stroke; including also stroke in subjects/patients with hypertension), traumatic brain injury, spinal cord injury, peripheral nerve injury, brain edema, or glioma, using a compound of formula (I) or a pharmaceutically acceptable salt or solvate thereof.

The neurological disease or disorder to be treated or prevented in accordance with the present invention, specifically the neurological disease or disorder involving an NKCC (preferably NKCC1), is not particularly limited, and is preferably selected from stroke (e.g., ischemic stroke; including, in particular, the use of the compounds according to the invention in promoting recovery after stroke, or the use of said compounds in reducing brain damage and/or neurological deficits after stroke), traumatic brain injury, spinal cord injury (including also chronic pain caused by spinal cord injury), peripheral nerve injury, brain edema, glioma (e.g., oligodendroglioma, ependymoma, subependymoma, choroid plexus papilloma, choroid plexus carcinoma, glioblastoma multiforme, astrocytoma, oligoastrocytoma, gliomatosis cerebri, or gliosarcoma), an autism spectrum disorder (e.g., autism, Asperger syndrome, childhood disintegrative disorder, or a pervasive developmental disorder as part of an autism spectrum disorder), Alzheimer's disease, schizophrenia, or Down syndrome (particularly mental disability in patients with Down syndrome). It is particularly preferred that the disease or disorder to be treated or prevented in accordance with the invention is stroke (particularly ischemic stroke), traumatic brain injury, spinal cord injury, peripheral nerve injury, brain edema, or glioma.

The present invention particularly relates to the treatment or prevention of stroke (preferably in a human subject/patient, who may be male or female, and particularly in a male human subject/patient).

The compound of formula (I) as well as the pharmaceutically acceptable salt or solvate thereof will be described in more detail in the following:

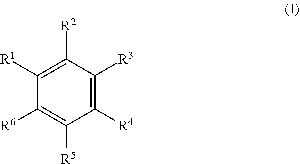

In formula (I), R¹ is selected from —(C₁₋₄ alkylene)-NH—(C₁₋₄ alkylene)-R¹¹ (e.g., —CH₂—NH—CH₂—R¹¹), —COO—(C₁₋₄ alkylene)-R¹¹ (e.g., —COO—CH₂—R¹¹), —O—CO—(C₁₋₄ alkylene)-R¹¹, —CO—(C₁₋₄ alkylene)-R¹¹, —CO—NH—(C₁₋₄ alkylene)-R¹¹ (e.g., —CO—NH—CH₂—R¹¹), —CO—N(C₁₋₄ alkyl)-(C₁₋₄ alkylene)-R¹¹, —NH—CO—(C₁₋₄ alkylene)-R¹¹ and —N(C₁₋₄ alkyl)-CO—(C₁₋₄ alkylene)-R¹¹, wherein R¹¹ is independently selected from —CF₃, —CN and halogen (e.g., —F, —Cl, —Br or —I). Preferably, R¹¹ is independently selected from —CF₃ and —CN; more preferably, R¹¹ is —CF₃. Specific examples of such R¹ groups include the corresponding groups R¹ of the compounds described in the examples section. It is particularly preferred that R¹ is —(C₁₋₄ alkylene)-NH—(C₁₋₄ alkylene)-CF₃, and even more preferably R¹ is —CH₂—NH—CH₂—CF₃.

R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —S H, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl).

Preferably, R² is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R² is hydrogen or C₁₋₄ alkyl. Even more preferably, R² is hydrogen.

R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). Preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one or more groups (particularly one group) independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). Even more preferably, R³ is selected from —SO₂—NH₂, —SO₂—NH(C₁₋₄ alkyl), —SO₂—N(C₁₋₄ alkyl)(C₁₋₄ alkyl), —SO₂—NH—(C₁₋₄ alkylene)-NH₂, —SO₂—NH—(C₁₋₄ alkylene)-NH(C₁₋₄ alkyl), —SO₂—NH—(C₁₋₄ alkylene)-N(C₁₋₄ alkyl)(C₁₋₄ alkyl), —SO₂—N═(C₁₋₄ alkylidene)—NH₂, —SO₂—N═(C₁₋₄ alkylidene)—NH(C₁₋₄ alkyl) and —SO₂—N═(C₁₋₄ alkylidene)—N(C₁₋₄ alkyl)(C₁₋₄ alkyl). Yet even more preferably, R³ is selected from —SO₂—NH₂, —SO₂—NH—CH₃, —SO₂—N(CH₃)₂, —SO₂—NH—(C₁₋₄ alkylene)-NH₂, —SO₂—NH—(C₁₋₄ alkylene)-NH—CH₃, —SO₂—NH—(C₁₋₄ alkylene)-N(CH₃)₂ (e.g., —SO₂—NH—CH₂CH₂—N(CH₃)₂), —SO₂—N═(C₁₋₄ alkylidene)—NH₂, —SO₂—N═(C₁₋₄ alkylidene)-NH—CH₃ and —SO₂—N═(C₁₋₄ alkylidene)-N(CH₃)₂ (e.g., —SO₂—N═CH—N(CH₃)₂). Still more preferably, R³ is —SO₂—NH₂.

R⁴ is selected from —O—R⁴¹, —S—R⁴¹, —NH—R⁴¹, —N(C₁₋₆ alkyl)-R⁴¹, halogen (e.g., —CI), hydrogen, carbocyclyl and heterocyclyl, wherein said carbocyclyl and said heterocyclyl are each optionally substituted with one or more (e.g., one, two or three) groups R⁴². Preferably, R⁴ is selected from —O—R⁴¹, —S—R⁴¹, —NH—R⁴¹, —N(C₁₋₆ alkyl)-R⁴¹, halogen, carbocyclyl and heterocyclyl, wherein said carbocyclyl and said heterocyclyl are each optionally substituted with one or more groups R⁴². More preferably, R⁴ is selected from —O—R⁴¹, —S—R⁴¹, —NH—R⁴¹, —N(C₁₋₆ alkyl)-R⁴¹, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². Even more preferably, R⁴ is selected from —O—R⁴¹, —S—R⁴¹, —NH—R⁴¹, —N(C₁₋₄ alkyl)-R⁴¹, aryl and heteroaryl, wherein said aryl and said heteroaryl are each optionally substituted with one or more groups R⁴².

R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³ Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴².

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R⁴ is selected from —O—(C₀₋₄ alkylene)-aryl, —O—(C₀₋₄ alkylene)-heteroaryl, —S—(C₀₋₄ alkylene)-aryl, —S—(C₀₋₄ alkylene)-heteroaryl, —NH—(C₀₋₄ alkylene)-aryl, —NH—(C₀₋₄ alkylene)-heteroaryl, —N(C₁₋₄ alkyl)—(C₀₋₄ alkylene)-aryl, —N(C₁₋₄ alkyl)—(C₀₋₄ alkylene)-heteroaryl, aryl and heteroaryl, wherein the aryl moiety of any of the aforementioned groups, the heteroaryl moiety of any of the aforementioned groups, said aryl and said heteroaryl are each optionally substituted with one or more (e.g., one, two or three) groups R⁴². Even more preferably, R⁴ is selected from —O-aryl, —O-heteroaryl, —S-aryl, —S-heteroaryl, —NH-aryl, —NH— heteroaryl, —N(C₁₋₄ alkyl)-aryl, —N(C₁₋₄ alkyl)-heteroaryl, aryl and heteroaryl, wherein the aryl moiety of any of the aforementioned groups, the heteroaryl moiety of any of the aforementioned groups, said aryl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Yet even more preferably, R⁴ is selected from —O-phenyl, —O-heteroaryl, —S-phenyl, —S-heteroaryl, —NH-phenyl, —NH-heteroaryl, —N(C₁₋₄ alkyl)-phenyl, —N(C₁₋₄ alkyl)-heteroaryl, phenyl and heteroaryl, wherein said heteroaryl or the heteroaryl moiety of any of the aforementioned groups is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein the phenyl moiety of any of the aforementioned groups, the heteroaryl moiety of any of the aforementioned groups, said phenyl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Still more preferably, R⁴ is —O-phenyl.

R⁵ is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹. Preferably, R⁵ is selected from —NH₂, —NH(C₁₋₆ alkyl), —N(C₁₋₆ alkyl)(C₁₋₆ alkyl) and —NO₂, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R⁵ is selected from —NH₂, —NH(C₁₋₆ alkyl), —N(C₁₋₆ alkyl)(C₁₋₆ alkyl), and —NO₂. Even more preferably, R⁵ is selected from —NH₂, —NH(C₁₋₆ alkyl), and —N(C₁₋₆ alkyl)(C₁₋₆ alkyl). Yet even more preferably, R⁵ is —NH(C₁₋₆ alkyl). Still more preferably, R⁵ is —NH—CH₂CH₂CH₂CH₃.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —S H, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl). Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

The compound of formula (I) may be, for example, any one of the specific compounds described in the examples section of this specification, either in non-salt form (e.g., free base/acid form) or as a pharmaceutically acceptable salt or solvate of the respective compound.

In particular, the compound of formula (I) may be a compound of any one of the following formulae, or a pharmaceutically acceptable salt or solvate thereof:

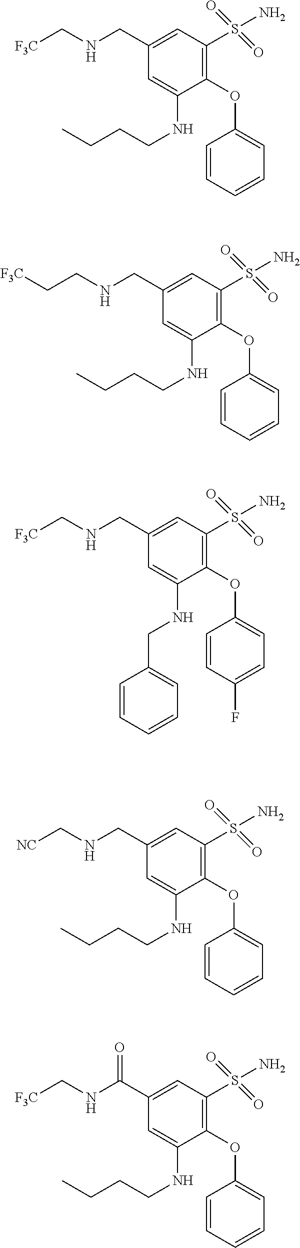 [SMILES: CCCCNc1cc(CNCC(F)(F)F)cc(S(N)(=O)=O)c1Oc1ccccc1 ; CCCCNc1cc(CNCCC(F)(F)F)cc(S(N)(=O)=O)c1Oc1ccccc1 ; NS(=O)(=O)c1cc(CNCC(F)(F)F)cc(NCc2ccccc2)c1Oc1ccc(F)cc1 ; CCCCNc1cc(CNCC#N)cc(S(N)(=O)=O)c1Oc1ccccc1 ; CCCCNc1cc(C(=O)NCC(F)(F)F)cc(S(N)(=O)=O)c1Oc1ccccc1]

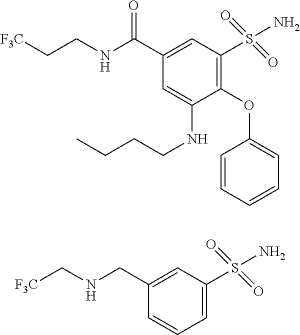 [SMILES: CCCCNc1cc(C(=O)NCCC(F)(F)F)cc(S(N)(=O)=O)c1Oc1ccccc1 ; NS(=O)(=O)c1cccc(CNCC(F)(F)F)c1]

For a person skilled in the field of synthetic chemistry, various ways for the preparation of the compounds of formula (I) will be readily apparent. For example, the compounds of formula (I) can be prepared in accordance with or in analogy to the synthetic routes described in the examples section.

The following definitions apply throughout the present specification, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₆ alkenyl” denotes an alkenyl group having 2 to 6 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more carbon-to-carbon double bonds. The term “C₂₋₆ alkynyl” denotes an alkynyl group having 2 to 6 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₁₅ alkylene” denotes an alkylene group having 1 to 15 carbon atoms, and the term “C₀₋₁₅ alkylene” indicates that a covalent bond (corresponding to the option “Co alkylene”) or a C₁₋₁₅ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkylidene” refers to a divalent acyclic hydrocarbon group which may be linear or branched, which is connected to the remainder of the respective compound via a double bond, and which does not comprise any other double bond (i.e., which does not comprise any double bond except for the one that connects the alkylidene group to the remainder of the respective compound) or any triple bond. An alkylidene group may, e.g., be attached to a carbon atom or to a nitrogen atom of the remainder of the respective compound. A “C₁₋₆ alkylidene” denotes an alkylidene group having 1 to 6 carbon atoms. Preferred exemplary alkylidene groups are methylidene (═CH₂), ethylidene (═CH—CH₃), propylidene (e.g., ═CH—CH₂CH₃ or ═C(—CH₃)—CH₃), or butylidene (e.g., ═CH—CH₂CH₂CH₃, ═C(—CH₃)—CH₂CH₃, or ═CH—CH(—CH₃)—CH₃). Unless defined otherwise, the term “alkylidene” preferably refers to C₁₋₄ alkylidene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl.

Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—CI), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of nucleotides in a given nucleic acid, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically or physiologically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically/physiologically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically/physiologically acceptable salt of the compound of formula (I) is a hydrochloride salt.

Moreover, the scope of the invention embraces the compounds of formula (I) in any solvated form, including, e.g., solvates with water (i.e., as a hydrate) or solvates with organic solvents such as, e.g., methanol, ethanol or acetonitrile (i.e., as a methanolate, ethanolate or acetonitrilate), or in any crystalline form (i.e., as any polymorph), or in amorphous form. It is to be understood that such solvates of the compounds of the formula (I) also include solvates of pharmaceutically acceptable salts of the compounds of the formula (I).

Furthermore, the compounds of formula (I) may exist in the form of different isomers, in particular stereoisomers (including, e.g., geometric isomers (or cis/trans isomers), enantiomers and diastereomers) or tautomers. All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor© HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the invention can be carried out, e.g., as described generally in the “Spray Drying Handbook”, 5th ed., K. Masters, John Wiley & Sons, Inc., NY (1991), in WO 97/41833, or in WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are topical administration, oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, particularly 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). However, the compound of formula (I) or a pharmaceutical composition comprising the compound of formula (I) can also be administered in combination with one or more further therapeutic agents, such as, e.g., one or more further therapeutic agents selected from phenobarbital, phenytoin, valproate (or valproic acid), carbamazepine, lamotrigine, levetiracetam, ethosuximide, and pharmaceutically acceptable salts of any of the aforementioned agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents (e.g., one or more of the corresponding exemplary therapeutic agents mentioned above) may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in one or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features described herein, including any combination of general and/or preferred features. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety.

More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention will now be described by reference to the following examples which are merely illustrative and are not to be construed as a limitation of the scope of the present invention.

### Examples

The compounds described in this section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### Example 1: Synthesis of Various Compounds According to the Invention

### General Methods

All chemicals and solvents were purchased from commercial suppliers (Sigma Aldrich, Merck, Apollo Scientific and TCI Europe) at analytical grade. Bumetanide was obtained from OChem Inc., Des Plaines, IL, US.

To monitor reactions via thin layer chromatography, silica gel F₂₅₄ coated aluminum sheets from Merck were used.

As a stationary phase for column chromatography silica gel 60 70-230 mesh ASTM from Merck was used.

Melting points were measured on a ThermoGalen Kofler hot stage microscope.

¹H- and ¹³C-NMR spectra were recorded on a Bruker Advance (200 and 50 MHz respectively) and chemical shifts are reported in ppm relatively to the solvent residual line or tetramethylsilane as internal standard.

Mass spectra were recorded on a Shimadzu (GC-17A; MS-QP5050A) spectrometer. The peak intensity is specified in percent relative to the biggest signal in the spectrum.

Elemental analysis were performed by Mag. Johannes Theiner at the University of Vienna and all reported values are within +/−0.4% of the calculated values.

### 3-(Butylamino)-5-(chloromethyl)-2-phenoxy-benzenesulfonamide (TEPS 76; reference)

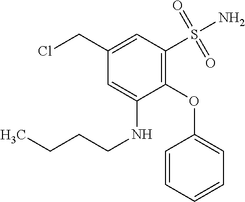 [SMILES: CCCCNc1cc(CCl)cc(S(N)(=O)=O)c1Oc1ccccc1]

1 mmol (0.35 g) of 3-(butylamino)-5-(hydroxymethyl)-2-phenoxy-benzenesulfonamide (Toellner K et al., Annals of Neurology (2014), 75(4), 550-562) was dissolved in 5 mL of thionyl chloride and heated to 80° C. for three hours. The thionyl chloride was evaporated under reduced pressure and the substance was vacuum-dried for one hour. The product was purified by recrystallization from 70% MeOH, yielding 0.34 g of brown crystals (92% yield). ¹H NMR (200 MHz, chloroform-d) δ 7.43-7-27 (m, 3H), δ 7.08 (t, J=7.3 Hz, 1H), δ 7.02-6.79 (m, 3H), δ 4.88 (s, 2H) δ 4.57 (s, 2H), δ 3.07 (t, J=6.9 Hz, 2H), δ 1.54-1.33 (m, 2H), δ 1.28-1.08 (m, 2H), δ 0.83, J=7.1 Hz (t, 3H). MS m/z: 368/370 M⁺

### 3-(Butylamino)-2-phenoxy-5-[(2,2,2-trifluoroethylamino)methyl]benzenesulfonamide (STS66)

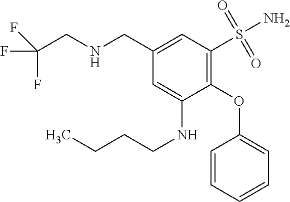 [SMILES: CCCCNc1cc(CNCC(F)(F)F)cc(S(N)(=O)=O)c1Oc1ccccc1]

### General Procedure A:

1 mmol (369 mg) of 3-(butylamino)-5-(chloromethyl)-2-phenoxy-benzenesulfonamide (TEPS 76) was dissolved in 3 mL dimethylformamide (DMF). To this 2 mmol (157 μl) of 2,2,2-trifluoroethylamine were added and the mixture was stirred at room temperature overnight. After the reaction was completed, which was verified by thin layer chromatography, the fluid was evaporated under reduced pressure, yielding a white crude product. This crude product was purified by column chromatography (ethyl acetate/petroleum ether 6+4) and recrystallization from 70% MeOH, yielding 130 mg of white crystals (30% yield). ¹H NMR (200 MHz, Methanol-d4) δ 7.34-7.18 (m, 3H), δ 7.09-6.96 (m, 2H), δ 6.94-6.83 (m, 2H), δ 3.90 (s, 2H), δ 3.29-3.17 (m, 2H), δ 3.09 (t, J=6.8 Hz, 2H), δ 1.49-1.32 (m, 2H), δ 1.26-1.06 (m, 2H), δ 0.81 (t, J=7.2 Hz, 3H). MS m/z: 431 M⁺

### 3-(Butylamino)-5-[(cyanomethylamino)methyl]-2-phenoxy-benzenesulfonamide (TEPS 13)

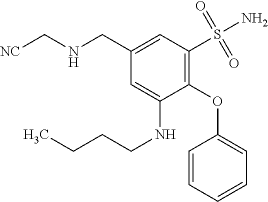 [SMILES: CCCCNc1cc(CNCC#N)cc(S(N)(=O)=O)c1Oc1ccccc1]

TEPS 13 was prepared according to general procedure A, but instead of 2,2,2-trifluoroethylamine, 1.2 mmol (167 μl) triethylamine and 1.2 mmol (71 μl) of aminoacetonitrile were added. The crude product was purified by column chromatography (EtOAc/petroleum ether 1+1) and recrystallization from 70% EtOH yielding 180 mg of beige powder (46% yield). ¹H NMR (200 MHz, chloroform-d) δ 7.40-7.22 (m, 3H), δ 7.16-6.85 (m, 4H), δ 5.02 (s, 2H), δ 3.91 (s, 2H), δ 3.88-3.78 (m, 1H), δ 3.60 (s, 2H), δ 3.20-3.00 (m, 2H), δ 2.99-2.83 (d, 1H), δ 1.49-1.34 (m, 2H), δ 1.26-1.12 (m, 2H), δ 0.90-0.74 (m, 3H). MS m/z: 388 M⁺

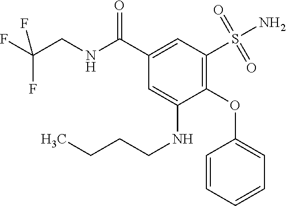 [SMILES: CCCCNc1cc(C(=O)NCC(F)(F)F)cc(S(N)(=O)=O)c1Oc1ccccc1]

To a solution of 1 mmol (364 mg) of Bumetanide in 5 mL dry tetrahydrofuran 1.2 mmol (194 mg) of 1,1-carbonyldiimidazole (CDI) were added and the mixture was stirred for two hours. Once TLC did not show any bumetanide remaining, 2 mmol (157 μl) of trifluoroethylamine were added and the mixture was stirred at room temperature overnight. Once the reaction was completed it was poured into 20 ml of 5% NaHCO₃ and extracted with ethyl acetate. The organic phase was then dried over Na₂SO₄ and the solvent was removed under reduced pressure. The crude product was then purified by recrystallization from EtOH to yield 159 mg of white powder (36% yield). ¹H NMR (200 MHz, Methanol-d4) δ 7.73 (d, J=2.1 Hz, 1H), 7.44 (d, J=2.1 Hz, 1H), 7.38-7.20 (m, 2H), 7.14-6.98 (m, 1H), 6.98-6.86 (m, 2H), 4.10 (q, J=9.3 Hz, 2H), 3.13 (t, J=6.8 Hz, 2H), 1.56-1.34 (m, 2H), 1.30-1.03 (m, 3H), 0.82 (t, J=7.2 Hz, 3H). MS m/z: 445 M⁺

### 3-[(2,2,2-Trifluoroethylamino)methyl]benzenesulfonamide

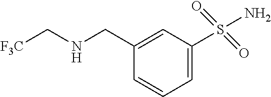 [SMILES: NS(=O)(=O)c1cccc(CNCC(F)(F)F)c1]

5 mmol (1.41 g) of 2,2,2-trifluoro-N-[(4-sulfamoylphenyl)methyl]acetamide (Augurusa, A., et al., 2016) were dissolved in 10 mL dry tetrahydrofuran (THF). The mixture was cooled at 0-4° C. and flooded with argon gas. 25 mmol (12.5 mL) of LiAlH₄ (2.0 M in THF) were added carefully in three portions every 30 minutes, then the solution was heated to 60° C. for 3 hours. The mixture was stirred overnight at room temperature. Again, the mixture was cooled at 0-4° C. and the reaction was quenched with 5% aqueous NH₄Cl. 2 N HCl was added until the mixture was completely clear and extracted two times with ethyl acetate. The aqueous phase was neutralized by adding 2 M NaOH and again extracted two times with ethyl acetate. The second organic phase was dried over sodium sulfate and evaporated under reduced pressure. Afterwards the product was recrystallized from isopropanol. The resulting product yielded 387 mg of white crystals (28.9% yield). ¹H NMR (200 MHz, DMSO-d₆) δ 7.79 (A-part of AB system, J_{AB}=8.3 Hz, 2H), 7.52 (B-part of AB system, J_{AB}=8.3 Hz, 2H), 7.31 (s, 2H), 3.86 (d, J=5.7 Hz, 2H), 3.32-3.11 (m, 2H), 3.09-2.96 (m, 1H). ¹³C NMR (50 MHz, DMSO-d₆) δ 144.3, 142.6, 126.2 (q, J=279.3 Hz), 128.1, 125.6, 51.8, 48.7 (q, J=30.3 Hz). MS m/z: 269 M⁺

### 3-(Benzylamino)-2-(4-fluorophenoxy)-5-[(2,2,2-trifluoroethylamino)methyl]benzenesulfonamide (TEPS 88)

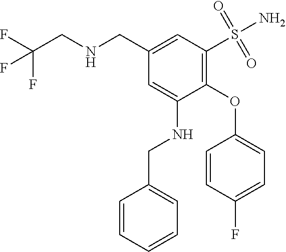 [SMILES: NS(=O)(=O)c1cc(CNCC(F)(F)F)cc(NCc2ccccc2)c1Oc1ccc(F)cc1]

### Step 1: 4-(4-fluorophenoxy)-3-nitro-5-sulfamoyl-benzoic acid (TEPS 84)

To a suspension of 20 mmol (5.61 g) of 4-chloro-3-nitro-5-sulfamoyl-benzoic acid (561 mg) (WO 2012/018635) in 30 mL water, 80 mmol NaHCO₃ (6.8 g) were added cautiously followed by 40 mmol (4.77 g) 4-fluorophenol. This solution was stirred at 85° for 16 hours. After cooling to room temperature, the precipitate was filtered off and dissolved in 10 mL of hot water. Then 6N HCl was added and the resulting precipitate was filtered off and dried to yield 4.35 g of a yellow solid (61% yield). ¹H NMR (200 MHz, DMSO) δ 14.01 (brs, 1H), 8.83-8.54 (m, 2H), 7.88 (s, 2H), 7.15 (t, J=8.8 Hz, 2H), 7.05-6.86 (m, 3H). ¹³C NMR (50 MHz, DMSO) δ 164.7, 158.56 (d, J=239.5 Hz), 153.25 (d, J=2.3 Hz), 148.2, 143.4, 140.3, 133.6, 130.9, 128.6, 118.24 (d, J=8.5 Hz), 116.56 (d, J=23.7 Hz). MS m/z 356

### Step 2: 3-amino-4-(4-fluorophenoxy)-5-sulfamoyl-benzoic acid (TEPS 85)

To an aqueous solution of LiOH (adjusted to pH 11) 10 mmol (3.56 g) TEPS84 and 350 mg palladium on activated charcoal (5% Pd/C) were added. The resulting mixture was hydrogenated at room temperature. When the H₂ uptake became negligible, the mixture was filtered and the filtrate was acidified with 6N HCl and extracted with ethyl acetate three times. The combined organic layers were washed with brine, dried over Na₂SO₄ and dried under reduced pressure to yield 2.15 g of a brown solid (66% yield). ¹H NMR (200 MHz, DMSO) δ 7.78-7.47 (m, 2H), 7.30 (s, 2H), 7.19-7.00 (m, 2H), 6.99-6.76 (m, 2H), 5.32 (s, 2H). ¹³C NMR (50 MHz, DMSO) δ 166.9, 157.8 (d, J=236.9 Hz), 152.8 (d, J=2.0 Hz), 143.2, 139.3, 138.3, 128.3, 120.7, 117.3 (d, J=8.4 Hz), 116.1, 115.7. MS m/z 326

### Step 3: Methyl 3-(benzylamino)-4-(4-fluorophenoxy)-5-sulfamoyl-benzoate (TEPS 86)

To a suspension of 2 mmol (652 mg) TEPS85 in 10 mL MeOH 5 mmol (0.6 mL) benzylbromide were added. The mixture was then refluxed for 16 hour to form a solution. After the reaction was completed, MeOH was removed under reduced pressure and 20 mL 5% NaHCO₃ were added. This mixture was extracted three times with ethyl acetate and the combined organic layers were washed with brine, dried over Na₂SO₄ and the solvent was removed under reduced pressure. The crude product was purified by column chromatography (ethyl acetate/petroleum ether 3+7) to yield 351 mg of a white solid (41% yield). ¹H NMR (200 MHz, DMSO) δ 7.67 (d, J=1.9 Hz, 1H), 7.40 (s, 2H), 7.35-7.04 (m, 8H), 6.96-6.80 (m, 2H), 6.21 (t, J=6.0 Hz, 1H), 4.35 (d, J=6.0 Hz, 2H), 3.81 (s, 3H). ¹³C NMR (50 MHz, DMSO) δ 165.8, 158.0 (d, J=237.2 Hz), 153.1 (d, J=2.0 Hz), 142.8, 140.5, 139.5, 138.3, 128.8, 127.3, 127.3, 127.1, 117.4 (d, J=8.3 Hz), 116.1, 115.7, 52.9, 46.2. MS m/z 430

### Step 4: 3-(Benzylamino)-2-(4-fluorophenoxy)-5-(hydroxymethyl)benzenesulfonamide (TEPS 87)

In a three necked flask 2 mmol of TEPS86 (860 mg) were dissolved in 8 mL anhydrous THF under argon atmosphere. Then 4 mL of a 1 M DIBAL-H solution in toluene were added. After one, two, three and four hours, respectively, another 2 mL of the 1 M DIBAL-H solution in toluene were added each time and the reaction was stirred overnight. After TLC showed no remaining TEPS86 the mixture was cooled to 0° C. and quenched with 5% aqueous NH₄Cl solution causing a gel-like substance to precipitate. The precipitate was then dissolved in 2 N HCl and extracted three times with ethyl acetate. The combined organic layers were washed three times with water, once with brine and dried over Na₂SO₄. The fluids were evaporated under reduced pressure and purified by recrystallization from ethanol to yield 665 mg of beige powder (83% yield). ¹H NMR (200 MHz, DMSO) δ 7.34-7.00 (m, 1 OH), 6.92-6.74 (m, 3H), 5.86-5.69 (m, 1H), 4.38 (s, 2H), 4.30 (d, J=5.2 Hz, 2H). ¹³C NMR (50 MHz, DMSO) δ 157.7 (d, J=236.5 Hz), 153.7, 140.6, 140.0, 137.4, 135.5, 128.7, 128.1, 127.2, 117.2 (d, J=8.1 Hz), 115.7 (d, J=23.3 Hz), 113.5, 112.6, 63.0, 46.3. MS m/z 402

### Step 5: 3-(Benzylamino)-2-(4-fluorophenoxy)-5-[(2,2,2-trifluoroethylamino)methyl]benzene-sulfonamide (TEPS 88)

1.5 mmol (604 mg) of TEPS87 were dissolved in 5 mL thionyl chloride and heated to 80 C.° for three hours. The thionyl chloride was evaporated under reduced pressure. The product was purified by column chromatography (ethyl acetate/petroleum ether 7+3) to yield 470 mg of brown solid (74% yield). 1 mmol (420 mg) of this intermediate benzyl chloride was dissolved in 5 mL of DMF, to this solution 2 mmol (157 μl) of 2,2,2-trifluoroethylamine were added and the mixture was stirred at room temperature overnight in a sealed vial. After the reaction was completed, which was verified by thin layer chromatography, the fluid was evaporated under reduced pressure. This crude product was purified by column chromatography (ethyl acetate/petroleum ether 3+7) and recrystallization from ethanol, yielding 86 mg of white crystals (18% yield). ¹H NMR (200 MHz, MeOD) δ 7.32-7.11 (m, 6H), 7.09-6.71 (m, 5H), 4.34 (d, J=3.8 Hz, 2H), 3.84 (d, J=26.0 Hz, 2H), 3.05 (q, J=9.8 Hz, 2H). ¹³C NMR (50 MHz, MeOD) δ 142.0, 139.0, 137.3, 128.1, 126.7, 116.5, 116.4, 115.5, 115.4, 115.0, 114.2, 52.1, 46.4. MS m/z 483

### 3-(Butylamino)-4-phenoxy-5-sulfamoyl-N-(3,3,3-trifluoropropyl)benzamide (TEPS 101)

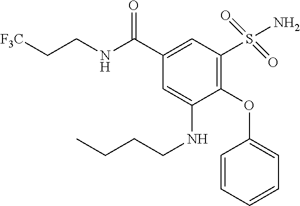 [SMILES: CCCCNc1cc(C(=O)NCCC(F)(F)F)cc(S(N)(=O)=O)c1Oc1ccccc1]

1 mmol (364 mg) of bumetanide was dissolved in in 5 mL dry tetrahydrofuran. 1.2 mmol (194 mg) 1,1-carbonyldiimidazole were added and the mixture was stirred for three hours. After the thin-layer chromatography showed that all bumetanide reacted, 2 mmol (300 mg) trifluoropropan-1-Amine were added and the mixture was stirred at room temperature overnight. After the reaction was completed 20 ml of 5% NaHCO₃ were added and it was extracted three times with ethyl acetate. The collected organic phase was washed with brine and dried over Na₂SO₄. The solvent was then removed under reduced pressure. The crude product was purified via recrystallization from EtOH. Yield: 220 mg (47%).

¹H NMR (200 MHz, MeOD) δ 7.74-7.61 (m, 2H), 7.40 (d, J=2.0 Hz, 1H), 7.29 (t, J=7.9 Hz, 2H), 7.10-7.03 (m, 2H), 6.96-6.85 (m, 2H), 3.64 (t, J=7.0 Hz, 2H), 3.12 (t, J=6.8 Hz, 2H), 2.70-2.37 (m, 2H), 1.42 (p, J=6.8 Hz, 2H), 1.24-1.05 (m, 2H), 0.81 (t, 3H). ¹³C NMR (50 MHz, MeOD) δ 169.1, 157.8, 144.0, 140.6, 138.4, 132.9, 130.7, 127.96 (d, J=276.2 Hz), 124.0, 116.6, 114.9, 114.5, 43.7, 34.60 (q, J=4.0 Hz), 34.03 (q, J=27.8 Hz), 32.0, 20.8, 14.0. MS m/z 459

### 3-(Butylamino)-2-phenoxy-5-[(3,3,3-trifluoropropylamino)methyl]benzenesulfonamide (TEPS 102)

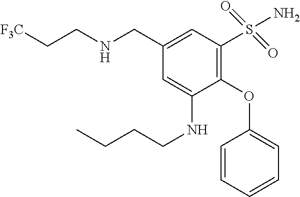 [SMILES: CCCCNc1cc(CNCCC(F)(F)F)cc(S(N)(=O)=O)c1Oc1ccccc1]

1.56 mmol (363 mg) of TEPS101 was dissolved in 20 mL of THF and 5.8 mmol (0.556 ml) borane dimethylsulfid complex was added. The reaction mixture was then stirred at 86° overnight. Once TLC showed that no starting material was present, the mixture was cooled to room temperature and then quenched with 20 ml of half-saturated aqueous NaHCO₃. It was extracted three times with 25 mL of ethyl acetate, washed brine and dried over Na₂SO₄. The solvent was removed under reduced pressure and the crude product was purified by column chromatography (ethyl acetate/petroleum ether and TEA, 1:1+20 mL of TEA) and recrystallized from 70% EtOH to yield 178 mg.(Yield 26%). ¹H NMR (200 MHz, CDCl₃) δ 7.38-7.16 (m, 3H), 7.06 (t, J=7.3 Hz, 1H), 6.91 (d, J=7.3 Hz, 3H), 4.90 (s, 1H), 3.79 (s, 2H), 3.06 (q, J=6.7 Hz, 2H), 2.91 (t, J=7.1 Hz, 2H), 2.34 (qt, J=10.9, 7.1 Hz, 2H), 1.41 (p, J=6.8 Hz, 2H), 1.17 (dq, J=13.7, 6.9 Hz, 2H), 0.82 (t, J=7.2 Hz, 3H). ¹³C NMR (50 MHz, CDCl₃) δ 156.4, 142.4, 138.2, 135.7, 135.6, 133.0, 130.1, 126.79 (d, J=276.8 Hz), 123.5, 120.5, 115.5, 115.3, 114.3, 53.4, 43.2, 42.23 (q, J=3.3 Hz), 34.41 (q, J=27.7 Hz), 31.2, 19.9, 13.8. MS m/z 445

### Example 2: NKCC1 Inhibitory Activity of the Compounds According to the Invention

The compounds of formula (I) according to the present invention are inhibitors of Na⁺—K⁺-2Cl⁻—cotransporters (NKCCs), particularly of NKCC1. The NKCC1 inhibitory activity of the compounds of the invention can be determined, for example, using the following NKCC1A activity assay.

To activate NKCC1A prior to the uptake experiment, hNKCC1A-expressing oocytes (Lykke, K., et al. 2016) or uninjected control oocytes are pre-incubated for 30 min at room temperature in a K⁺-free solution. To measure K⁺ influx, oocytes are exposed to an isosmotic test solution in which KCl is substituted for choline chloride and ⁸⁶Rb⁺ is added as a tracer for K⁺. Bumetanide (positive control), a compound of formula (I) according to the invention (“drug”), or control vehicle (negative control) are added to the test solution. The uptake assay is then performed at room temperature with mild agitation for 5 min. The influx experiments are terminated and the radioactivity present is determined by liquid scintillation β-counting with Opti-Fluor scintillation using a Liquid Scintillation Analyzer. hNKCC1A-mediated K⁺ uptake is then assessed as ([flux_{NKCCl-expressing oocytes} in presence of ×μM drug]−[flux_{uninjected oocytes} in presence of ×μM drug]), in order to correct for endogenous NKCC activity. A reduction in hNKCC1A-mediated K⁺ uptake observed with a test compound is indicative of the compound inhibiting NKCC1. When the exemplary compounds of formula (I) described in Example 1 are subjected to this assay, it can be confirmed that they exhibit NKCC1 inhibitory activity.

### Example 3: Effects of the Compounds According to the Invention on Reducing Brain Damage and Neurological Deficits after Ischemic Stroke in Mice

### Introduction

Stimulation of the WNK-SPAK/OSR1 kinases and their substrate Na⁺—K⁺-2Cl⁻ cotransporter 1 (NKCC1) play critical roles in cerebral edema and neurological functional deficits after ischemic stroke. Either NKCC1 inhibitor bumetanide (BMT) or knockout of WNK3 or SPAK shows profound protective effects in a mouse model of ischemic stroke. In this study, the efficacy of two pharmacological inhibitors of NKCC1, i.e. the novel NKCC1 inhibitor STS66 which is an exemplary compound of formula (I) according to the present invention, as well as the lipophilic BMT prodrug STS5 (reference compound), on reducing ischemic stroke-induced brain damage in mouse were investigated.

### Material and Methods

### Materials

Bumetanide (BMT) and AngII were from Sigma (Sigma-Aldrich, St Louis, Missouri, USA). STS5 is described in Erker et al., 2016 (where it is referred to as BUM5); and STS66 is described in Example 1 above.

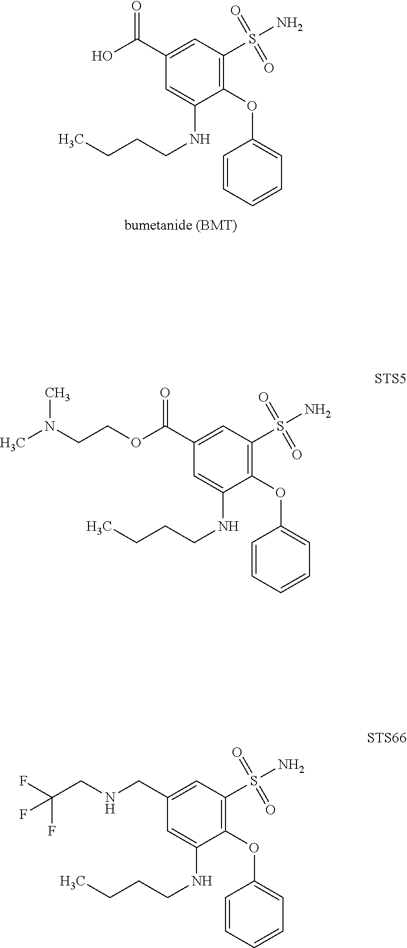 [SMILES: CCCCNc1cc(C(=O)O)cc(S(N)(=O)=O)c1Oc1ccccc1 ; CCCCNc1cc(C(=O)OCCN(C)C)cc(S(N)(=O)=O)c1Oc1ccccc1 ; CCCCNc1cc(CNCC(F)(F)F)cc(S(N)(=O)=O)c1Oc1ccccc1]

### Animals

All animal experiments were approved by the University of Pittsburgh Institutional Animal Care and Use Committee and performed in accordance with the National Institutes of Health Guide for the Care and Use of Laboratory Animals. The manuscript adheres to the ARRIVE guidelines for reporting animal experiments. Wild type C57BL/6j genetic background mice at 8-14 weeks old (26-30 g body weight, male and female) were used in the study. Animals were housed in a temperature- and humidity-controlled animal facility with a 12-hour light-dark cycle. Food and water were available ad libitum.

Transient Focal Cerebral Ischemia Model (tMCAO)

Transient focal cerebral ischemia was induced in mice by intraluminal occlusion of the left middle cerebral artery (MCA) for 50 min as described previously (Chen H et al., 2005). Mice were anesthetized with 3% isoflurane in 70%: 30% N₂O/O₂ until they were unresponsive to the tail pinch test. Animals were then fitted with a nose cone blowing 1.5% isoflurane for anesthesia maintenance. The left common carotid artery was exposed and the occipital artery branches of the external carotid artery were isolated and coagulated. The internal carotid artery was isolated and the extracranial branch was dissected and ligated. A rubber silicon-coated monofilament suture (6-0) was introduced into the internal carotid artery lumen and gently advanced approximately 8-9 mm to block the MCA blood flow for 50 min. The rectal temperature was maintained at 37.0±0.5° C. during surgery through a temperature-controlled heating pad. Achievement of ischemia was confirmed by monitoring regional cerebral blood flow (rCBF) with laser speckle contrast imager (Pericam). For reperfusion, the suture was withdrawn after the MCAO. The incision was closed and the mouse recovered under a heating lamp to maintain the core temperature (36.0-37.0° C.) during the 30-60 min recovery period. After recovery, animals were returned to their cages with free access to food and water.

Permanent Focal Cerebral Ischemia Model (pdMCAO)

Permanent focal cerebral ischemia was induced by permanent occlusion of the distal middle cerebral artery (pdMCAO) in mice (Suenaga J, et al., 2015). Under anesthesia as described above, a skin incision at the midline of the neck was made. After being separated from the vagal nerve, the left CCA was exposed and occluded by ligation and the skin was sutured. Another skin incision (1 cm) was made between the left eye and the ear using fine operation scissors. The temporal muscle was identified and detached from the skull in its apical and dorsal part without totally removing the muscle by using the forceps. The MCA below the transparent skull in the rostral part of the temporal area, dorsal to the retro-orbital sinus was identified. If the MCA bifurcation is not visible (due to an anatomical normal variation), the vessel most rostral was identified. The skull above the MCA branch was thinned out with the drill until it has a thin and translucent texture. The artery, proximal and distal to the MCA bifurcation, was coagulated with the electrocoagulation forceps (in a bipolar mode at 7 W). The temporal muscle was relocated to its position and the burr hole was covered with wax and the skin wound was sutured and infiltrated with analgesia bupivacaine (100 μl 0.25%) topically. The animal was placed in a cage and monitored for recovery from anesthesia.

### Drug Treatment

Vehicle: DMSO (2 ml/kg body weight/day), the NKCC1 inhibitor bumetanide (BMT, 10 mg/kg body weight/day in DMSO), bumetanide's prodrug STS5 (13 mg/kg body weight/day in DMSO), or the novel NKCC1 inhibitor STS66 according to the invention (12 mg/kg body weight/day in DMSO) was administered via intraperitoneal injection (i.p). The initial half dose of the drugs (BMT, STS5, STS66) was given at 3 h after the onset of reperfusion and the second half dose at 8 h reperfusion in the tMCAO model.

Anpiotensin II (AnpII) infusion-induced hypertension

Mice received 14 days of infusion of either saline or AngII via osmotic minipumps (model 1002; Alzet, Cupertino, California, USA) implanted subcutaneously in the intrascapular region under isofluorane anesthesia. Sterile procedures were used to prevent postoperative infection at the site of implantation. The pumps were loaded either with saline for the vehicle (Veh control) group, or the Ang II peptide at a rate of 1000 ng/kg/min as described previously (Nagai M, et al., 2011; Lu H, et al., 2015). The pumps were removed after 14 days of infusion.

### Neurological Function Tests

Neurological functional deficits in mice were assessed in a blinded manner with the following tests: neurological score, adhesive tape removal test, corner test, cylinder test, and grid walking foot-fault test. These tests are established for identifying and quantifying sensorimotor deficits and postural asymmetries (Bederson J B, et al., 1986; Zhang L, et al., 2002; Schaar K L, et al., 2010).

### Neurological Score:

Neurological deficit grading system was used to evaluate neurological deficit at 1, 2, 3, 5, 7, 10 and 14 days after tMCAO as described previously (Bederson J B et al,1986).

### Corner Test:

Neurological functional deficits in mice were determined by the corner test. The apparatus consists of two cardboards (each size is 30 cm×20 cm) placed together at a 30° angle to form a narrow alley. The mouse was placed between the two angled boards facing the corner. When exiting the corner, uninjured mice will turn left or right randomly.

After tMCAO, animals with unilateral brain damage will exhibit unidirectional turning. The numbers of left and right turns of each mouse during 10 trials were recorded, and turning movements that were not part of a rearing movement were not scored (Zhang L, et al., 2002).

### Adhesive Tape Removal Test:

An adhesive tape removal test was used to measure somatosensory deficits. Two pieces of adhesive tape (4 mm×3 mm) were attached to the forepaws in an alternating sequence and with equal pressure by the experimenter before each trial. The removal time is defined as the time at which the animal removes the tape. The trial ended after the adhesive patch was removed or after 2 min had elapsed. Pre-operative training was carried twice per day for three days and were tested on day 1, 2, 3, 5, 7, 10 and 14 days after tMCAO (Bouet V, et al., 2009).

### Grid Walking Foot-Fault Test:

The grid walking test is sensitive to deficits in descending motor control. Each mouse was placed on a stainless steel grid floor (20×40 cm with a mesh size of 4 cm²) elevated 1 m above the floor. Every animal was tested for three 1-min trials. The data were expressed as the number of foot fault errors made by the forelimbs contralateral to the injured hemisphere as a percentage of total steps (Jun Zhang, et al., 2017).

### Brain Infarct Volume and Swelling Measurement

At 24 h post-reperfusion, mice were anesthetized with 5% isoflurane and then decapitated. Coronal brain slices (2 mm thickness) were stained with 1% 2,3,5-triphenyltetrazolium chloridemonohydrate (TTC) for 20-30 min and brain slices were scanned (Begum G et al., 2015). Ischemic lesions were traced in each slice in a blinded manner, and the total volume of infarction was calculated with correction for edema, as described by Swanson et al., 1990 using ImageJ software. Infarct areas were summed across all slices and multiplied by slice thickness to yield total infarct volume (mm³). Brain swelling was determined with the following formula. For tMCAO: swelling (% contralateral hemispheric volume)=[(ipsilateral hemispheric volume)−(contralateral hemispheric volume)]/(contralateral hemispheric volume)×100, as described before (Guo Q G et al., 2009; Sunghee Cho et al., 2005).

### Statistics

The animal subjects were randomly assigned into different studies and surgical procedures, and data analyses were performed by investigators who were blinded to the experimental conditions. Values are expressed as means SD or SEM. Statistical analysis was performed using the multiple comparisons, ANOVA multiple comparisons test (Graphpad, Prism7 software). A p-value less than 0.05 was considered statistically significant.

### Results

Efficacy of BMT, STS5 and STS66 on Reducing Brain Infarction and Edema in Normotensive Mice after Ischemic Stroke

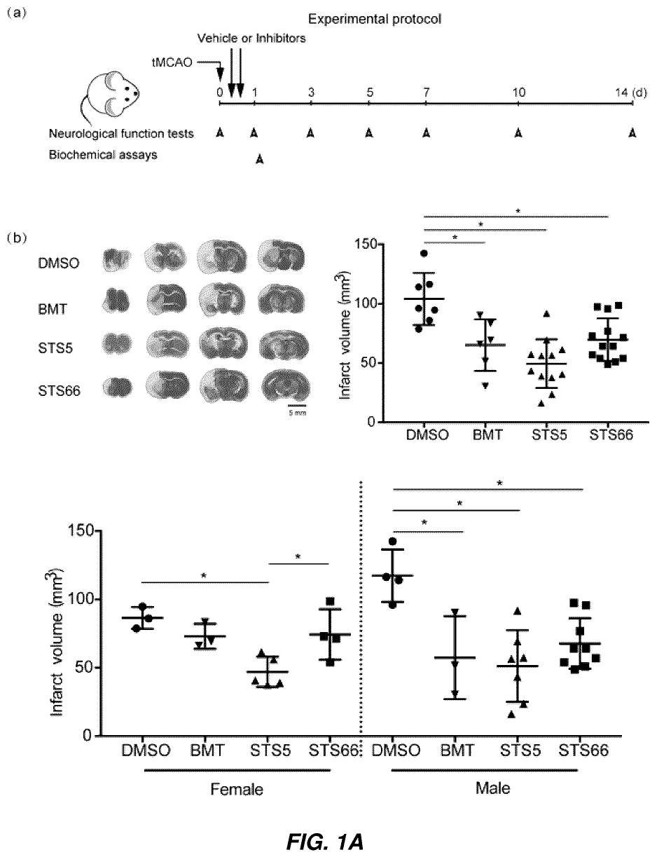

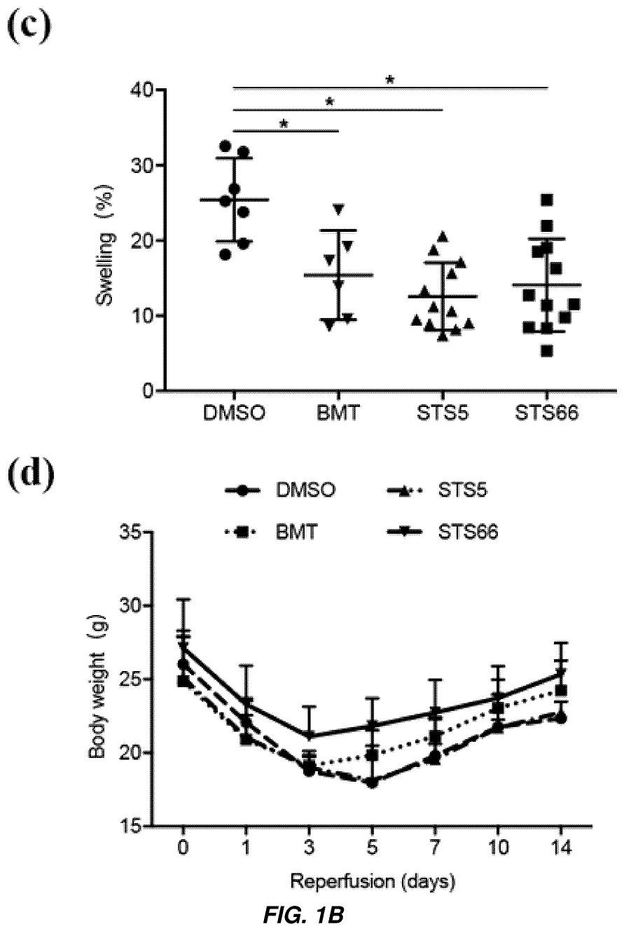

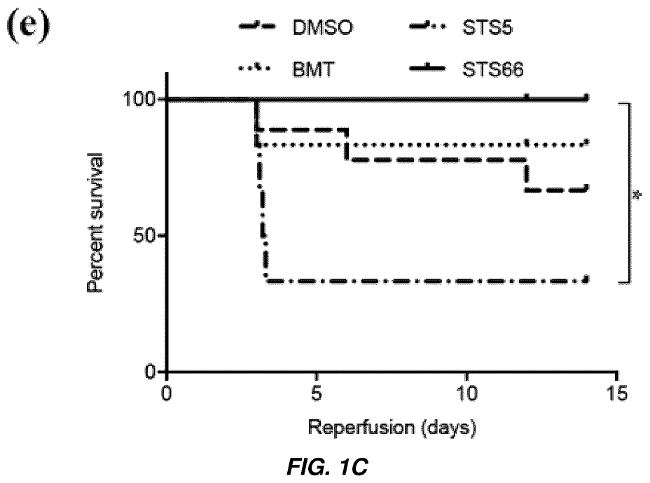

Ischemic stroke in C57B6/j mice via tMCAO and post-stroke administration of vehicle DMSO control, BMT, STS5 or STS66 were performed as illustrated in FIG. 1A. Brain infarct volume and swelling were measured at 24 h after reperfusion. Ischemia stroke led to 104.1±21.8 mm³ infarction and 25.4±5.5% hemispheric edema in the vehicle control mice (see FIGS. 1B and 1C). In the BMT treatment group, the infarct volume was reduced to 65.2±21.7 mm³, and edema to 15.4±5.9% (p<0.05, vs. Veh Control). The STS5-treated mice also exhibited smaller infarct volume (49.5±20.6 mm³) and lest edema (12.6±4.5%, p<0.05 vs. Veh control). In the case of STS66, the infarct volume was 69.7±17.9 mm³, and edema was 14.1±6.1%, which was also significantly less than that in the vehicle-treated mice. However, there were no statistically significant differences among the BMT-, STS5-, and STS66-treated mice either for infarct volume or cerebral edema. The effects of these NKCC1 inhibitors were then examined in male and female mice. As shown in FIG. 1B, the infarct volume in male mice showed similar results as the combined data. However, in the female mice, the STS5 treatment exhibited the smallest infarct volume (46.9±5.0 mm³, n=5), which is significantly different from the STS66-treated mice (74.3±9.2 mm³, n=4).

Animal body weight and animal survival rates were also monitored among four groups during 1-14 days after tMCAO. As demonstrated in FIG. 1D, no differences in the body weight changes were detected in the DMSO Veh control and BMT-, STS5-, STS66-treated mice after tMCAO. As shown in FIG. 1E, the mortality rate of the Veh control animals between 1-14 days post tMCAO was ˜33.3%. The BMT-treated mice show ˜16.7% mortality. But, an increase in mortality was detected in the STS5-treated mice after stroke (˜66.7%), which is significantly higher than the Veh control group. Interestingly, none of the STS66-treated mice died after tMCAO. These findings strongly indicate that STS66 is a superior NKCC1 inhibitor candidate for ischemic stroke therapy development.

Effects of BMT and STS66 on Improving Neurological Function in Mice after Ischemic Stroke

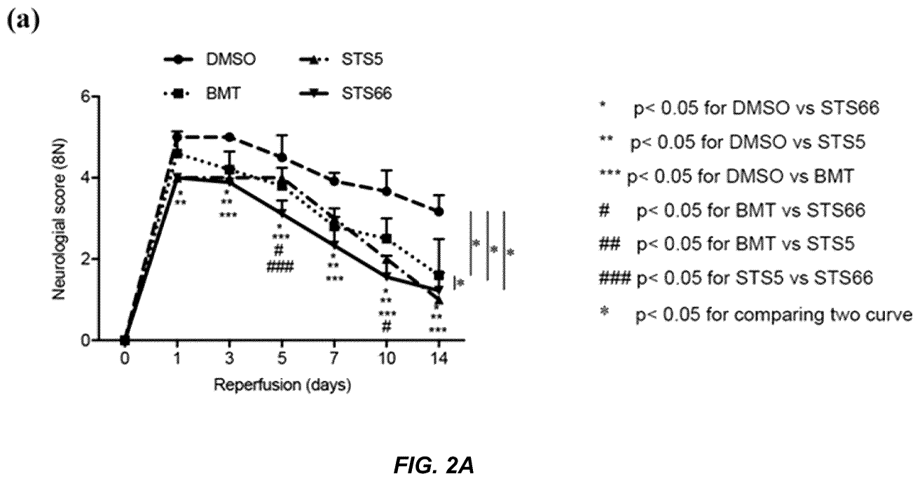

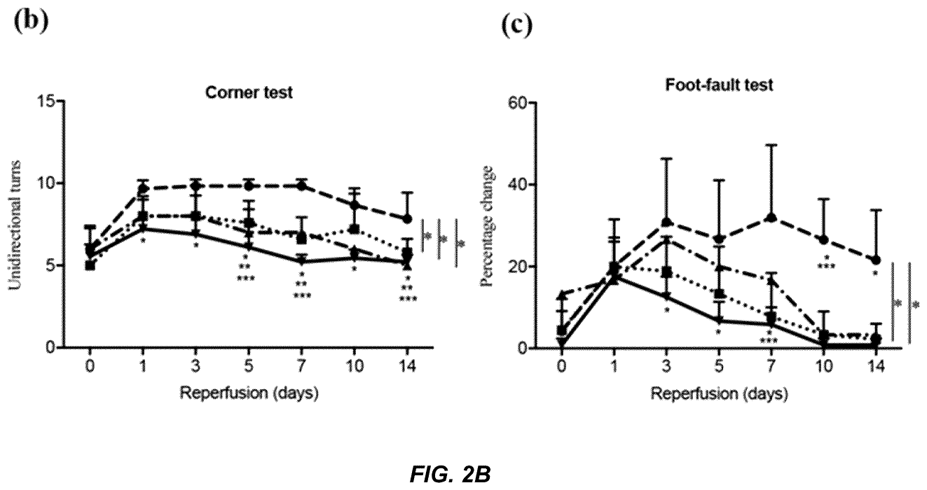

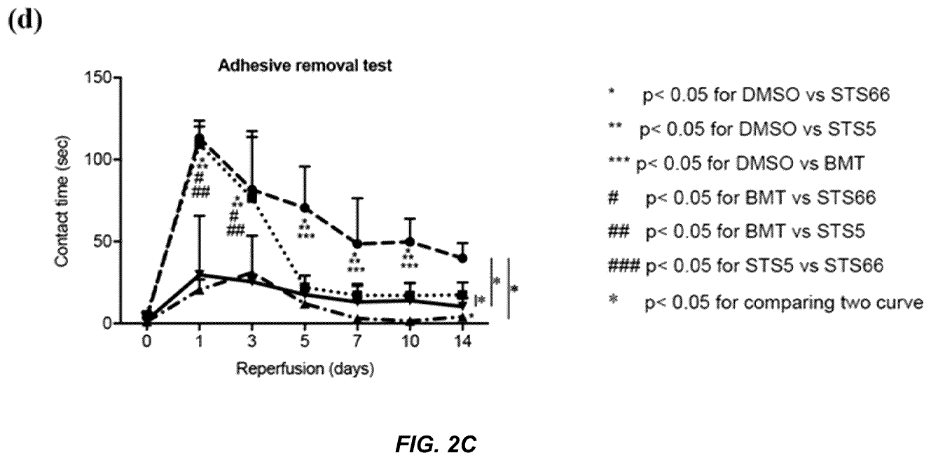

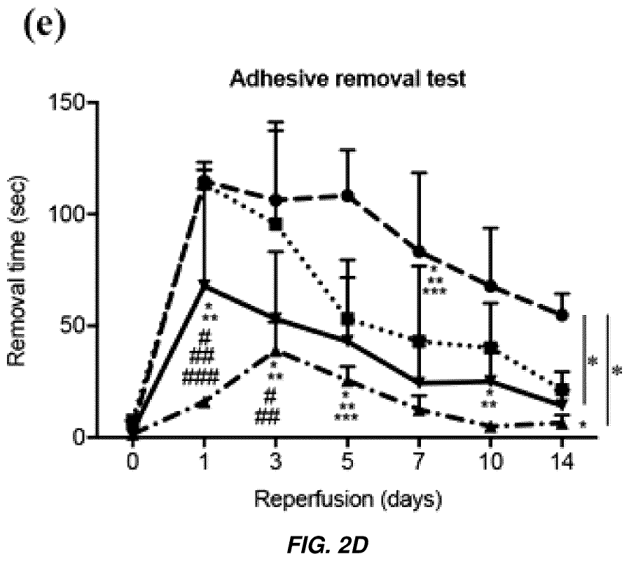

A series of neurological behavioral tests were conducted to assess changes of sensorimotor function deficits in mice treated with either BMT or STS66 following ischemic stroke. According to 1-14 days monitor and behavior test, BMT and STS66 treatments improved all sensorimotor deficits in normotensive mice after stroke. Both BMT- and STS66-treated mice exhibited progressive decrease in neurological deficit score. Importantly, a faster improvement of neurological deficit scores was detected in the STS66-treated groups, from day 1 to day 14 post-reperfusion (neurological score 4.0±0.0 to 1.2±0.4), as also shown in FIG. 2A. STS66 works the best (p<0.0001). The BMT-treated mice show better outcomes than the Veh control mice (neurological score of 4.6±0.5 to 3.2±0.4), BMT treatment also shows a significant difference to the STS66-treated mice at day 5 (3.8±0.4) and day 10 (2.5±0.5). Both BMT and STS66 treatment exhibited neuroprotective effects (see FIG. 2B), and STS66 shows definite superior effects at every testing day in the corner test (p<0.05). At day 3 post-stroke, the foot-fault rate of the STS66-treated mice was 12.5±2.5%, significantly different from the Veh control mice (30.8±15.5%, p<0.05). The adhesive contact and removal tests revealed that the Veh control mice required a significant longer time to complete the tasks at day 1 to day 3 post-stroke (see FIG. 2D), followed by a slow recovery from day 5 to 7 post-stroke. In contrast, the BMT- or STS66-treated mice performed faster in the adhesive tape removal (STS66-treated mice for 29.7±36.0 s to 10.3±5.2 s), significantly shorter than the Veh control mice (113.2±10.6 s to 39.8±9.3 s) or the BMT-treated mice (109.4±10.8 s to 17.4±7.6 s), as shown in FIG. 2D. Taken together, blockade of NKCC1 protein with BMT, STS5, or STS66 displayed different degrees of neuroprotective effects after ischemic stroke. STS66 was found to be superior over both BMT and STS5.

Efficacy of STS66 on Preventing AngII-Induced Hypertensive Mice from Developing Worsened Infarct and Cerebral Edema after Ischemic Stroke

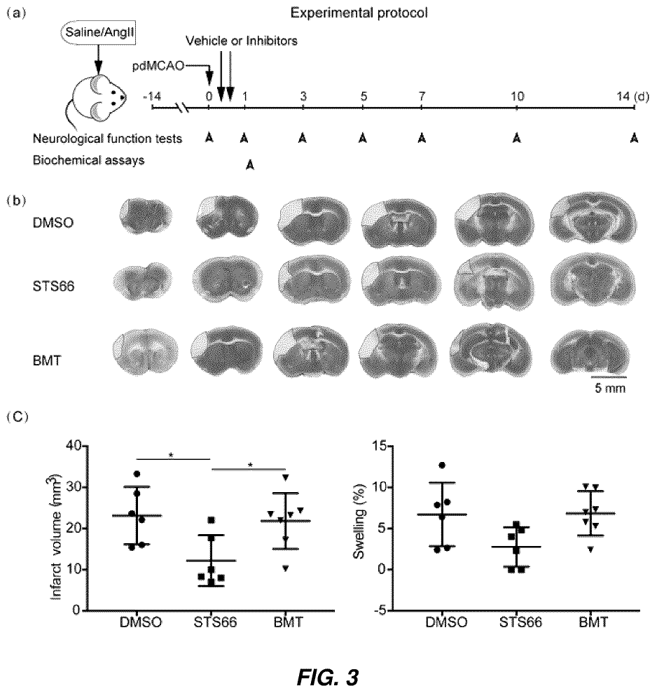

FIG. 3A shows the experimental protocol of induction of hypertension in mice via osmotic pump infusion of AngII (1000 ng/kg/min) for 14 days. The efficacy of NKCC1 inhibitors was then tested in AngII-induced hypertensive mice after permanent MCAO model (pdMCAO), which is more clinically relevant to the majority ischemic stroke patients (McBride D W, et al., 2017). Brain infarct volume and swelling were measured at 24 h after pdMCAO. The DMSO Veh control, BMT, or STS66 was then administered in mice via i.p. with the first half dose at 3 h after pdMCAO and the second half dose at 8 h after pdMCAO. As shown in FIGS. 3B and 3C, BMT treatment had similar infarction (22.4±2.9 mm³) and swelling (7.1±1.1%), not significantly different compared to the Veh Control. But the STS66-treated group showed much smaller infarct volume (13.3±2.5 mm³) and less brain edema (2.4±1.1%) at 24 h post-pdMCAO (see FIG. 3C). These data clearly demonstrate that STS66 is also highly effective in reducing ischemic damage in AngII-mediated hypertensive mice after permanent focal ischemia without reperfusion (pdMCAO stroke).

These findings show that the compounds of formula (I), including in particular the compound STS66, are particularly well suited for the treatment of stroke, as reflected by the observed reduction of brain infarction and cerebral swelling after stroke, as well as a considerably improved therapeutic outcome, including improved survival and improved sensorimotor functional recovery after stroke. The observed neuroprotective effect further supports a prophylactic therapy (prevention) of stroke. The compounds of formula (I) can hence advantageously be used for the treatment or prevention of stroke as well as other neurological diseases/disorders involving NKCCs, such as traumatic brain injury, spinal cord injury, peripheral nerve injury, brain edema, or glioma.

### Example 4: Effects of the Compounds According to the Invention on Glioma

### Materials and Methods

### Materials

Bumetanide (BMT, #B3023) and Temozolomide (TMZ, #T2577) were purchased from Sigma-Aldrich (St. Louis, MO). Dulbecco's Modified Eagle Medium (DMEM/HEPES, Cat #12430-054) and Penicillin/streptavidin (Cat #15240062) were from Gibco (Carlsbad, CA). Fetal bovine serum (FBS) was obtained from Invitrogen (Carlsbad, CA). Anti-phospho-NKCC1(Thr206) antibody was developed by Dr. Yang (Taiwan National University) (Moriguchi et al. 2005; Yang et al. 2010). Monoclonal antibody against total NKCC (T4) was from the Developmental Studies Hybridoma Bank (Iowa City, IA). Antibody against a-tubulin (Cat #2125), rabbit antibody against Ki67 (Cat #9129S) and antibody against cleaved caspase-3 (Cat #9661S) were from Cell Signaling (Beverly, MA). Goat antibody against NKCC1 (Cat #ab99558) was from Abcam Ltd (Cambridge, MA). BCA Protein Assay Kit (Cat #23227) was from Thermo Scientific (Rockford, IL).

### Cell Cultures and Authentication

Immunogenic mouse glioma GL26 and non-immunogenic mouse SB28-GFP glioma cells were used as previously described (Kohanbash et al. 2017). GL26 and SB28-GFP glioma cells obtained from Prof. Gary Kohanbash, PhD, were derived as described previously (Kosaka et al. 2014) and maintained in DMEM/HEPES containing 10% heat-inactivated FBS, 2 mM L-glutamine, 1× Penicillin/streptavidin and 1 mM sodium pyruvate. Cultures were passaged approximately every 4 days with fresh medium at a density of 106 cells/75 cm2 in a culture flask. Passage 10-30 of glioma cells were used in the study. All cell lines were authenticated by short tandem repeat (STR) DNA fingerprinting (by IDEXX BioResearch, Columbia, MO) in the past 6 months. In addition, PCR analysis was performed to confirm the absence of mycoplasma infection in all cell cultures.

### Rubidium Uptake Assay

GL26 and SB28-GFP cells were seeded in 24-well plates and the rubidium uptake assay was performed on cells that were 60% confluent. TMZ was added to cells for 48 hr incubation. The medium was then removed from the wells and washed with wash buffer (Rb⁺ free). After wash buffer was aspirated, isotonic and hypertonic solutions (contain Rb⁺) with BMT were added, and cells were incubated at 37° C. for 5 min. After this incubation period, cells were washed with isotonic or hypertonic solutions (Rb⁺ free). After washing, cell lysis buffer (200 μl/well) was added to the plate to release intracellular Rb⁺. The Rb⁺ concentration was measured using an automated atomic absorption spectrophotometer (Ion Channel Reader, ICR-8000; Aurora Biomed, Vancouver, Canada).

### BrdU Proliferation Assay

Cell proliferation of GL26 and SB28-GFP cells was measured by quantifying BrdU incorporation. GL26 cells (5×103 cells/well) or SB28-GFP cells (1×103 cells/well) were seeded in 96-well plates in 100 μL media. After 24 hours in culture, cells were incubated with fresh medium plus the following reagents: DMSO vehicle (Con-Veh), BMT (B, 10 μM), TMZ (100 μM), or TMZ+B for 48 h. BrdU was added in the last 4 h period of the whole 48 h incubation. The incorporation of BrdU into newly synthesized DNA of proliferating cells was detected by using a peroxidase-conjugated antibody which reacts with the thymidine analogue BrdU. Bound anti-BrdU-peroxidase conjugated antibody was measured by a substrate reaction, and then quantified colorimetrically by an ELISA plate reader (Spectra MAX 190, Molecular Devices, Sunnyvale, CA) at dual wavelength of 450/550 nm.

### Immunoblotting

GL26 and SB28-GFP cells were washed with ice-cold PBS and incubated in RIPA buffer containing 1 pill of phosSTOP and 2 mM protease inhibitors as described before (Algharabli et al. 2012). Cells were lysed by sonication at 4° C. Protein content of the cellular lysate was determined with BCA Protein Assay Kit. Samples and sample buffer (Thermo Scientific, Rockford, IL, USA) were boiled at 95° C. for 5 min. The samples were then electrophoretically separated on 10% SDS gels. After transferring to PVDF membranes, the blots were blocked in 10% nonfat dry milk in TBS-T (Tris-buffered saline, 0.05% Tween-20) for 1 hour at room temperature and then incubated with appropriate primary antibodies (pNKCC1, 1:300 and tNKCC1, 1:3000) at 4° C. overnight. After rinsing with TBS-T, the blots were incubated with horseradish peroxidase-conjugated secondary IgG (1:2000) for 1 hr at RT. Bound antibody was visualized with an enhanced chemiluminescence assay. Protein band signal intensities were analyzed using ImageJ and normalized to α-tubulin expression.

### Mouse Syngeneic Glioma Model

All animal experiments were approved by the University of Pittsburgh Institutional Animal Care and Use Committee and performed in accordance with the National Institutes of Health Guide for the Care and Use of Laboratory Animals.

Six to eight-week-old female albino C57BL/6 mice were anesthetized with 2% isoflurane. Once in the anesthetic plane, mice were mounted on a stereotactic frame and an 1 cm incision was made along the midline of the cranium to expose the skull. Using a precision power drill with a fine tip needle, a single hole was made on the skull in right hemisphere (coordinates from bregma: +0.5 mm AP, +2.1 mm ML, and −3.2 mm DV). 1×105 GL26 or 4×104 GL26-mCitrine or 0.5×105 SB28-GFP cells (in 2 μL of serum-free DMEM) were injected into the right striatum in 4 min (at a rate of 0.5 μL/min), using a micro-pump injector and a 5-μl Hamilton syringe equipped with a 33-gauge needle. Cells were allowed to settle for 5 min and the needle was withdrawn slowly. The incision was closed with surgical staples. Ketofen (2 mg/kg, i.p.) was administrated once prior to surgery and daily for two days after the surgery and then daily if animals exhibit signs of pain. Animals were then allowed to recover in their cages under a heat lamp and access to water and wet chow.

### Drug Treatment Regimens

Starting 7 days after tumor cell implantation (d.p.i.), mice were randomly assigned to each treatment group and received the therapy for 5 consecutive days: vehicle control (1.25% DMSO in PBS, 10 ml/kg/day, i.p.), NKCC1 inhibitor BMT (B, 5 mg/kg, twice a day, i.p.), TMZ therapy (50 mg/kg/day, once a day, i.p.), or TMZ+NKCC1 inhibitor BMT (T of 50 mg/kg/day+B of 5 mg/kg, twice a day, i.p.) combination treatment.

### Animal Survival Test

Overall survival was evaluated in all mice. Tumor bearing animals were monitored daily for signs of pain, discomfort or neurological impairment. Signs of chronic pain, such as hunched posture, weight loss, absence of grooming behavior, and of neurological impairment, like seizures, weakness, difficulty walking, an inability to right themselves, circling behavior, and unusual aggressiveness or timidity were used to infer tumor development. In tumor cell injected mice, a loss of 20% body weight, severe neurological impairment, or major loss in body scoring index (<2.0 on a 5-point scale) were used as the humane endpoint. All other surviving mice were sacrificed at 90 days after glioma cell injection.

### Results

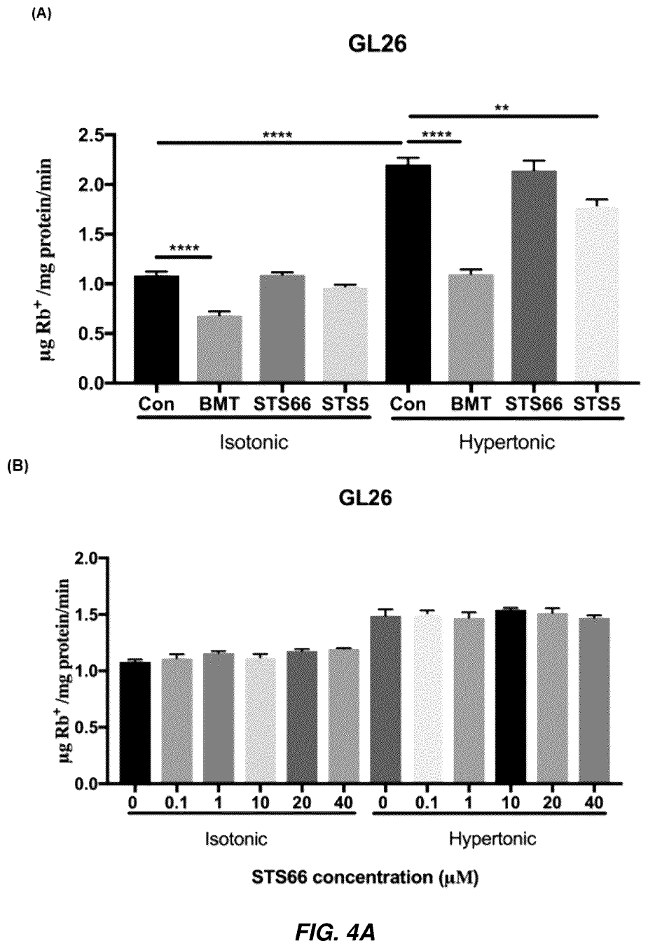

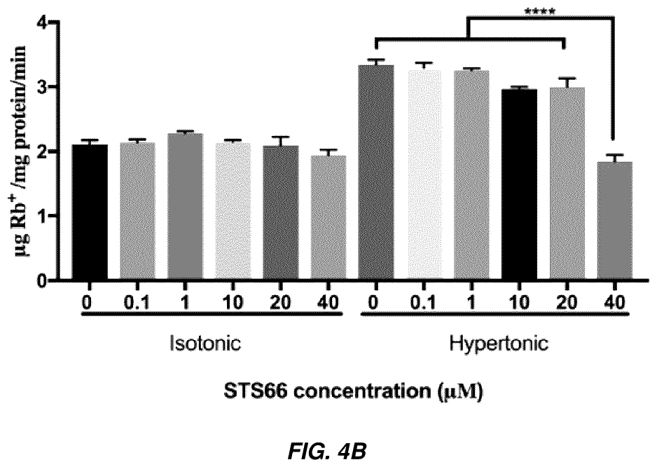

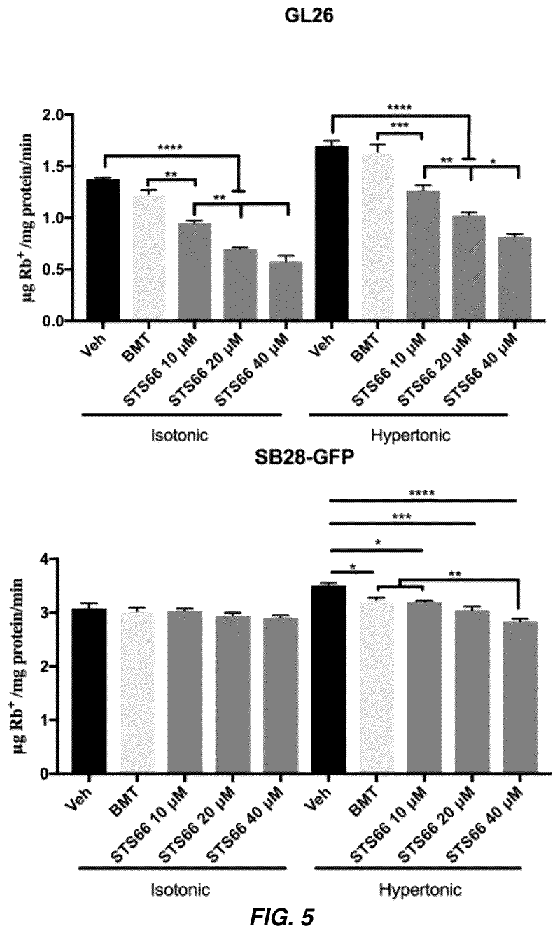

The results of these experiments are shown in FIGS. 4 to 8. In particular, it was found that STS66 does not inhibit Rb⁺ influx when drugs are incubated for short time (see FIG. 4). Notably, STS66 inhibited the Rb⁺ influx better than BMT when drugs were incubated for 48 h (see FIG. 5).

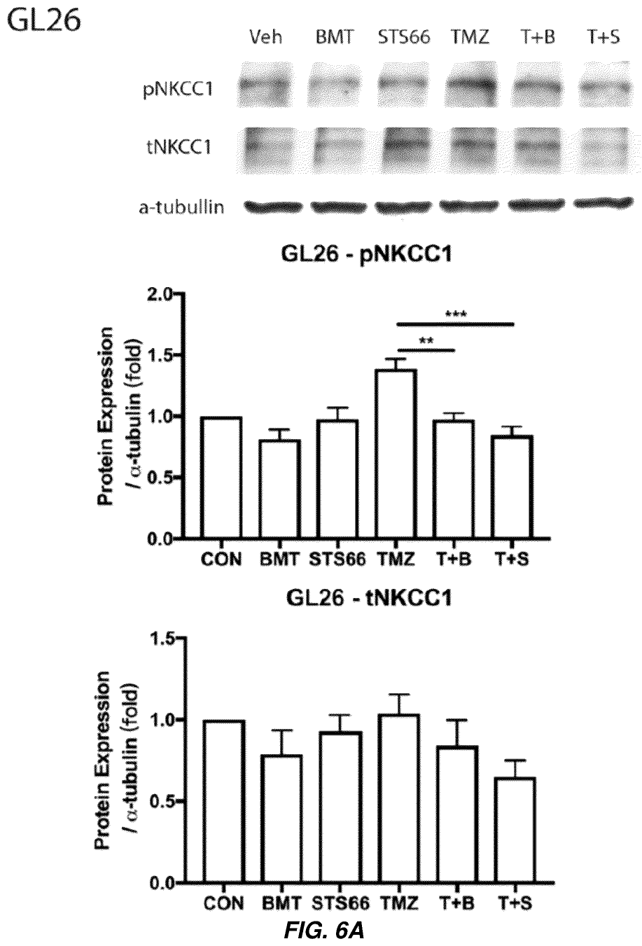

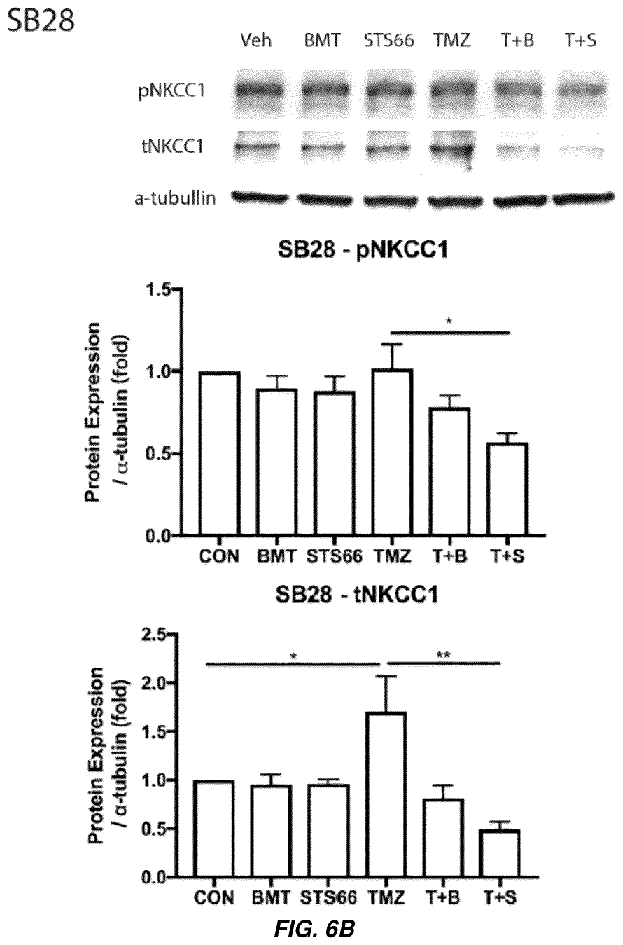

pNKCC1 and tNKCC1 expression did not show significant decrease when treated with STS66 alone compared to BMT. TMZ did not trigger the NKCC1 upregulation. However, the combination treatment T+S significantly decreased the expression of NKCC1 (see FIG. 6).

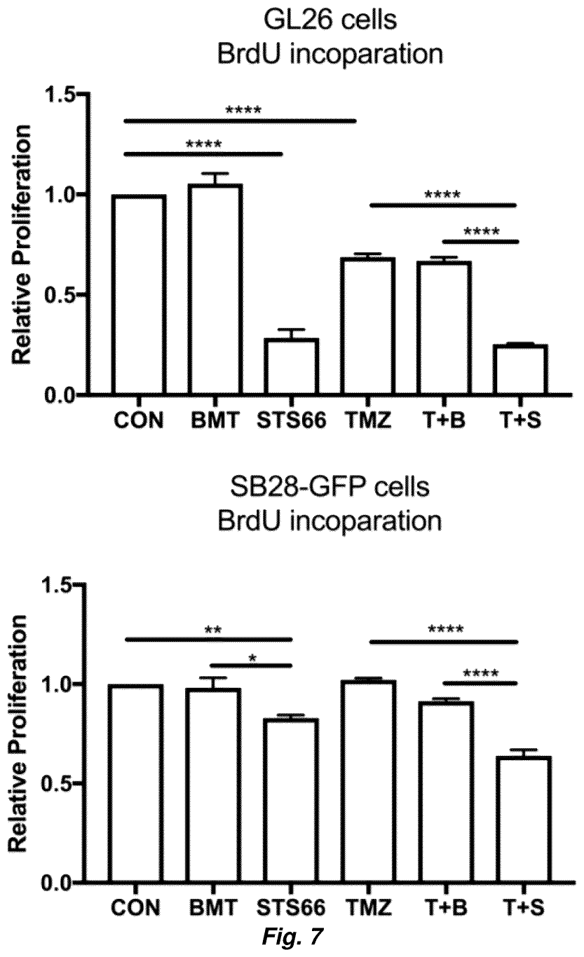

In the BrdU proliferation assay, STS66 showed more inhibition of GL26 and SB28-GFP cell proliferation than BMT (see FIG. 7).

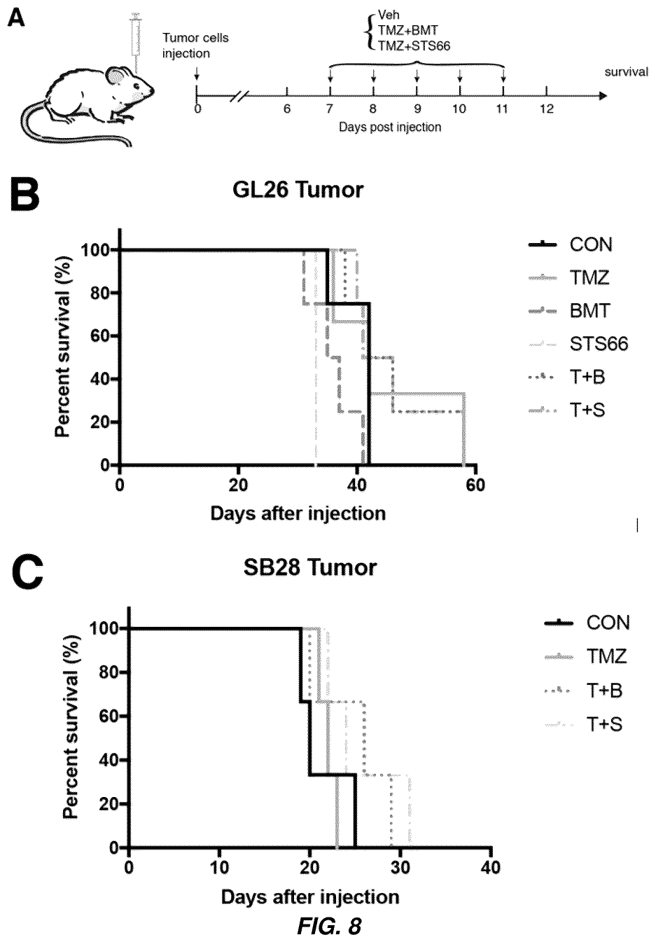

When the combinatorial regimen of BMT, STS66 and TMZ was tested in glioma bearing mice, it was found that T+S did not improve the survival of the mice, as shown in FIG. 8.

These results indicate that the compounds of formula (I), including in particular STS66, are suitable for the therapy of glioma and exhibit a more pronounced activity than bumetanide.

### REFERENCES

Alessi, D., Zhang, J., Khanna, A., Hochdorfer, T., Shang, Y., & Kahle, K. T. (2014). The WNKSPAK/OSR1 pathway: Master regulator of cation-chloride cotransporters. Science Signaling(334).
Algharabli J, Kintner D B, Wang Q W, Begum G, Clark P A, Yang S S, Lin S H, Kahle K T, Kuo J S, Sun D D. (2012) Inhibition of Na+-K+-2Cl(−) Cotransporter isoform 1 Accelerates Temozolomide-mediated Apoptosis in Glioblastoma Cancer Cells. Cellular Physiology and Biochemistry 30:33-48.
Allegaert K, Lahav A, van den Anker J N. (2016) Erratum to: A Mechanism to Explain Ototoxicity in Neonates Exposed to Bumetanide: Lessons to Help Improve Future Product Development in Neonates. Paediatr Drugs 18:475.
Ares G., Caceres P., Ortiz P. (2011) Molecular regulation of NKCC2 in the thick ascending limb. Am J Physiol Renal Physiol 301:F1143-F1159
Aronica E, Boer K, Redeker S, Spliet W G, van Rijen P C, Troost D, Gorter J A. (2007) Differential expression patterns of chloride transporters, Na+—K+-2Cl−—cotransporter and K+-Cl−—cotransporter, in epilepsy-associated malformations of cortical development. Neuroscience 145:185-96.
Augurusa, A., Mehta, M., Perez, M., Zhu, J., Stephan, D. (2016) Catalytic Reduction of Amides by Electrophilic Phosphonium Cations via FLP Hydrosilylation. The Royal Society of Chemistry 52: 12195-12198.
Bederson J B, Pitts L H, et al. (1986 May-June) Rat middle cerebral artery occlusion: evaluation of the model and development of a neurologic examination. Stroke. 17(3):472-6.
Begum G, Yuan H, Kahle K T, Li L, Wang S, Shmukler B E, Yang S S, Lin S H, Alper S L, Sun D (2015) Inhibition of WNK3 kinase signaling reduces brain damage and accelerates neurological recovery following ischemic stroke. Stroke 46(7): 1956-1965.
Bouet V, Boulouard M, Toutain J, et al. (2009) The adhesive removal test: a sensitive method to assess sensorimotor deficits in mice. Nat Protoc 4: 1560-1564.
Campbell S L, Robel S, Cuddapah V A, Robert S, Buckingham S C, Kahle K T, Sontheimer H. (2015) GABAergic disinhibition and impaired KCC2 cotransporter activity underlie tumor-associated epilepsy. Glia 63:23-36.
Chen H, Luo J, Kintner D B, et al. (2005) Na(.)-dependent chloride transporter (NKCC1)-null mice exhibit less gray and white matter damage after focal cerebral ischemia. J Cereb Blood Flow Metab; 25: 54-66.
Cho S, Eun-Mi Park, et al. (2005) Obligatory role of inducible nitric oxide synthase in ischemic preconditioning. Journal of Cerebral Blood Flow & Metabolism 25, 493-501
Cuddapah V A, Sontheimer H. (2011) Ion channels and tranporters in cancer. 2. Ion channels and the control of cancer cell migration. American Journal of Physiology-Cell Physiology 301:C₅₄₁-C_{549.}
Deidda, G., et al. (2015) Reversing excitatory GABAAR signaling restores synaptic plasticity and memory in a mouse model of Down syndrome. Nature Medicine 21, 318-326.
Deng Y., et al. (2016) Progress in Drug Treatment of Cerebral Edema. Mini-Reviews in Medicinal Chemistry 16: 917-925
Dzhala, V. I., Talos, D. M., Sdrulla, D. A., Brumback, A. C., Mathews, G. C., Benke, T. A., &Staley, K. J. (November 2005). NKCC1 transporter facilitates seizures in the developing brain. Nature Medicine(11), pp. 1205-1213.
Erker T, Brandt C, Töllner K, Schreppel P, Twele F, Schidlitzki A, Löscher W (2016) The bumetanide prodrug BUM5, but not bumetanide, potentiates the antiseizure effect of phenobarbital in adult epileptic mice. Epilepsia 57(5): 698-705
Garzon-Muvdi T, Schiapparelli P, ap Rhys C, Guerrero-Cazares H, Smith C, Kim D H, Kone L, Farber H, Lee D Y, An S S, et al. (2012) Regulation of Brain Tumor Dispersal by NKCC1 Through a Novel Role in Focal Adhesion Regulation. Plos Biology 10.
Guo Q, Wang G, Liu X, et al. (2009) Effects of gemfibrozil onoutcome after permanent middle cerebral artery occlusion in mice. Brain Res 1279: 121-130.
Haas B R, Sontheimer H. (2010) Inhibition of the Sodium-Potassium-Chloride Cotransporter Isoform-1 reduces glioma invasion. Cancer Res 70:5597-606.
Habela C W, Ernest N J, Swindall A F, Sontheimer H. (2009) Chloride accumulation drives volume dynamics underlying cell proliferation and migration. J Neurophysiol 101:750-7.
Hadjikhani N., et al. (2015) Improving emotional face perception in autism with diuretic bumetanide: A proof-of-concept behavioral and functional brain imaging pilot study. Autism Vol. 19(2) 149-157
Huang Y., et al. (2016) Acute spinal cord injury (SCI) transforms how GABA affects nociceptive sensitization. Experimental Neurology 285, 82-95
Kahle, K. T., & Staley, K. J. (2008, September). The bumetanide-sensitive Na—K-2Cl cotransporter NKCC1 as a potential target of a novel mechanism-based treatment strategy for neonatal seizures. Neurosurg Focus, pp. 1-7.
Kohanbash G, Carrera D A, Shrivastav S, Ahn B J, Jahan N, Mazor T, Chheda Z S, Downey K M, Watchmaker P B, Beppler C and others. (2017) Isocitrate dehydrogenase mutations suppress STAT1 and CD8+ T cell accumulation in gliomas. J Clin Invest 127:1425-1437.
Kosaka A, Ohkuri T, Okada H. (2014) Combination of an agonistic anti-CD40 monoclonal antibody and the COX-2 inhibitor celecoxib induces anti-glioma effects by promotion of type-1 immunity in myeloid cells and T-cells. Cancer Immunology Immunotherapy 63:847-857.
Lu, H., et al. (2015) Subcutaneous Angiotensin II Infusion using Osmotic Pumps Induces Aortic Aneurysms in Mice. J Vis Exp, 103.
Lykke, K., et al. (2016) The search for NKCC1-selective drugs for the treatment of epilepsy: Structure-function relationship of bumetanide and various bumetanide derivatives in inhibiting the human cation-chloride cotransporter NKCC1A. Epilepsy & Behavior, 59:42-49
Ma H, Li T, Tao Z, Hai L, Tong L, Yi L, Abeysekera I R, Liu P, Xie Y, Li J and others. (2019) NKCC1 promotes EMT-like process in GBM via RhoA and Rac1 signaling pathways. J Cell Physiol 234:1630-1642.
Maa, E. H., Kahle, K. T., Walcott, B. P., Spitz, M. C., & Staley, K. J. (2011). Diuretics and epilepsy: Will the past and present meet? Epilepsia(52), pp. 1559-1669.
MacKenzie G, O'Toole K K, Moss S J, Maguire J. (2016) Compromised GABAergic inhibition contributes to tumor-associated epilepsy. Epilepsy Research 126:185-196.
Markadieu, N., Delpire, E. (2014) Physiology and pathophysiology of SLC12A1/2 transporters Pflugers Arch—Eur J Physiol 466:91-105
McBride, D. W. and Zhang, J. H. (2017) Precision Stroke Animal Models: the Permanent MCAO Model Should Be the Primary Model, Not Transient MCAO. Transl Stroke Res.
Merner, N. D., et al. (2016) Gain-of-function missense variant in SLC12A2, encoding the bumetanidesensitive NKCC1 cotransporter, identified in human schizophrenia. Journal of Psychiatric Research 77 22-26
Merner, N. D., et al. (2015) Regulatory domain or CPG site variation in SLC12A5, encoding the chloride transporter KCC2, in human autism and schizophrenia. Frontiers in Cellular Neurosience 9, 386
Munoz, A., DeFelipe, J., & Alvarez-Leefmans, F. J. (2007). Cation-Chloride Cotransporters and GABAergic Innervation in the Human Epileptic Hippocampus. Epilepsia(48), pp. 663-673.
Moriguchi T, Urushiyama S, Hisamoto N, Iemura S I, Uchida S, Natsume T, Matsumoto K, Shibuya H. (2005) WNK1 regulates phosphorylation of cation-chloride-coupled cotransporters via the STE20-related kinases, SPAK and OSR1. Journal of Biological Chemistry 280:42685-42693.
Nagai, M., et al., (2011) Role of blood cell-associated angiotensin II type 1 receptors in the cerebral microvascular response to ischemic stroke during angiotensin-induced hypertension. Exp Transl Stroke Med, 3: p. 15.
Owens, D. F., & Kriegstein, A. R. (September 2002). Is there more to GABA than synaptic inhibition. Nature Reviews Neuroscience(3), pp. 715-727.
Payne, J. A., Rivera, C., Voipio, J., & Kaila, K. (April 2003). Cation-chloride co-transporters in neuronal communication, development and trauma. Trends in Neurosciences(26), pp. 199-206.
Peti-Peterdi J., Harris R. (2010) Macula densa sensing and signaling mechanisms of renin release. J Am Soc Nephrol 21:1093-1096
Schaar K L, Brenneman M M, Savitz S I. (2010) Functional assessments in the rodent stroke model. Exp Transl Stroke Med. 2(1):13. doi: 10.1186/2040-7378-2-13.
Schiapparelli P, Guerrero-Cazares H, Magana-Maldonado R, Hamilla S M, Ganaha S, Goulin Lippi Fernandes E, Huang C H, Aranda-Espinoza H, Devreotes P, Quinones-Hinojosa A. (2017) NKCC1 Regulates Migration Ability of Glioblastoma Cells by Modulation of Actin Dynamics and Interacting with Cofilin. EBioMedicine 21:94-103.
Suenaga, J., et al., (2015) White matter injury and microglia/macrophage polarization are strongly linked with age-related long-term deficits in neurological function after stroke. Exp Neurol. 272: p. 109-19.
Swanson R A, Morton M T, Tsao-Wu G, et al. (1990) A semiautomated method for measuring brain infarct volume. J Cereb Blood Flow Metab; 10: 290-293.
Töllner K, Brandt C, Töpfer M, et al. (2014) A novel prodrug-based strategy to increase effects of bumetanide in epilepsy. Ann Neurol; 75:550-562.
Tyzio R. et al. (2014) Oxytocin-Mediated GABA Inhibition During Delivery Attenuates Autism Pathogenesis in Rodent Offspring Science 343 (6171), 675-679)
Xu W., et al. (2016) Chloride Co-transporter NKCC1 Inhibitor Bumetanide Enhances Neurogenesis and Behavioral Recovery in Rats After Experimental Stroke. Mol Neurobiol DOI 10.1007/si12035-016-9819-0)
Yang S S, Lo Y F, Wu C C, Lin S W, Yeh C J, Chu P L, Sytwu H K, Uchida S, Sasaki S, Lin S H. (2010) SPAK-Knockout Mice Manifest Gitelman Syndrome and Impaired Vasoconstriction. Journal of the American Society of Nephrology 21:1868-1877.
Younus I, Reddy D S. (2018) A resurging boom in new drugs for epilepsy and brain disorders. Expert Rev Clin Pharmacol 11:27-45.
Zhang L, Schallert T, et al. (2002) A test for detecting long-term sensorimotor dysfunction in the mouse after focal cerebral ischemia. J Neurosci Methods. 117(2):207-14.
Zhang J, Pu H, et al. (2017) Inhibition of Na+—K+-2Cl− cotransporter attenuates blood brain barrier disruption in a mouse model of traumatic brain injury. Neurochem Int. 111:23-31.

1. A method of treating or preventing a neurological disease or disorder involving a Na⁺—K⁺-2Cl⁻—cotransporter (NKCC), comprising administering to a patient in need thereof a therapeutically effective amount of a compound of formula (I): 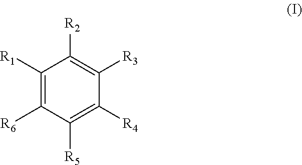
wherein:
R¹ is selected from —(C₁₋₄ alkylene)-NH—(C₁₋₄ alkylene)-R¹¹, —COO—(C₁₋₄ alkylene)-R¹¹, —O—CO—(C₁₋₄ alkylene)-R¹¹, —CO—(C₁₋₄ alkylene)-R¹¹, —CO—NH—(C₁₋₄ alkylene)-R¹¹, —CO—N(C₁₋₄ alkyl)—(C₁₋₄ alkylene)-R¹¹, —NH—CO—(C₁₋₄ alkylene)-R¹¹ and —N(C₁₋₄ alkyl)-CO—(C₁₋₄ alkylene)-R¹¹;
R¹¹ is independently selected from —CF₃, —CN and halogen;
R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —S H, —S(C₁₋₆ alkyl), —NH₂, —NH(C₁₋₆ alkyl), —N(C₁₋₆ alkyl)(C₁₋₆ alkyl), C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl);
R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl);
R⁴ is selected from —O—R⁴¹, —S—R⁴¹, —NH—R⁴¹, —N(C₁₋₆ alkyl)-R⁴¹, halogen, hydrogen, carbocyclyl and heterocyclyl, wherein said carbocyclyl and said heterocyclyl are each optionally substituted with one or more groups R⁴²;
R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more groups R⁴³;
each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl);
each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl);
R⁵ is selected from —NH₂, —NH(C₁₋₆ alkyl), —NO₂ and hydrogen, wherein the alkyl moiety of said —NH(C₁₋₆ alkyl) is optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹;
each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl); and
R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)—SO₂—(C₁₋₆ alkyl);
or a pharmaceutically acceptable salt or solvate thereof.

2. The method of claim 1, wherein R¹ is —(C₁₋₄ alkylene)-NH—(C₁₋₄ alkylene)—CF₃.

3. The method of claim 1, wherein R² is hydrogen.

4. The method of claim 1, wherein R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), and further wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl).

5. The method of claim 1, wherein R³ is —SO₂—NH₂.

6. The method of claim 1, wherein R⁴ is selected from —O-aryl, —O— heteroaryl, —S-aryl, —S-heteroaryl, —NH-aryl, —NH-heteroaryl, —N(C₁₋₄ alkyl)-aryl, —N(C₁₋₄ alkyl)-heteroaryl, aryl and heteroaryl, and wherein said aryl and heteroaryl moiety are each optionally substituted with one or more groups independently selected from the group consisting of C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

7. The method of claim 1, wherein R⁴ is —O-phenyl.

8. The method of claim 1, wherein R⁵ is selected from —NH₂, —NH(C₁₋₆ alkyl), and —NO₂.

9. The method of claim 1, wherein R⁵ is —NH—CH₂CH₂CH₂CH₃.

10. The method of claim 1, wherein R⁶ is hydrogen.

11. The method of claim 1, wherein the compound of formula (I) is selected from the group consisting of: 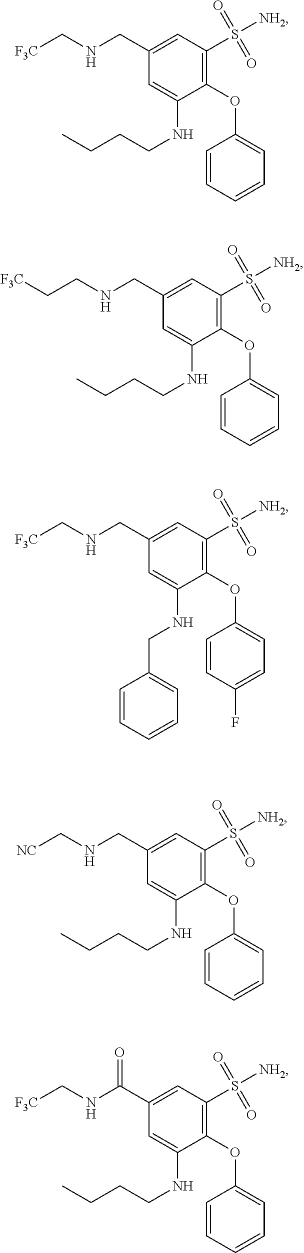 [SMILES: CCCCNc1cc(CNCC(F)(F)F)cc(S(N)(=O)=O)c1Oc1ccccc1 ; CCCCNc1cc(CNCCC(F)(F)F)cc(S(N)(=O)=O)c1Oc1ccccc1 ; NS(=O)(=O)c1cc(CNCC(F)(F)F)cc(NCc2ccccc2)c1Oc1ccc(F)cc1 ; CCCCNc1cc(CNCC#N)cc(S(N)(=O)=O)c1Oc1ccccc1 ; CCCCNc1cc(C(=O)NCC(F)(F)F)cc(S(N)(=O)=O)c1Oc1ccccc1] 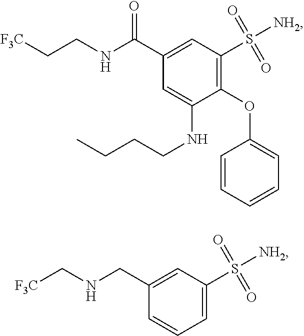
and pharmaceutically acceptable salts or solvates thereof.

12. The method of claim 1, wherein the compound of formula (I) is 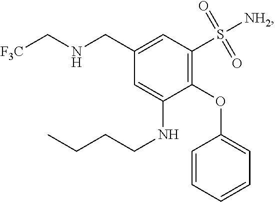 [SMILES: CCCCNc1cc(CNCC(F)(F)F)cc(S(N)(=O)=O)c1Oc1ccccc1]
or a pharmaceutically acceptable salt or solvate thereof.

13. The method of claim 1, wherein the neurological disease or disorder involving an NKCC is selected from the group consisting of stroke, traumatic brain injury, spinal cord injury, peripheral nerve injury, brain edema, glioma, an autism spectrum disorder, Alzheimer's disease, schizophrenia, and Down syndrome.

14. The method of claim 1, wherein the neurological disease or disorder involving an NKCC is selected from the group consisting of stroke, traumatic brain injury, spinal cord injury, peripheral nerve injury, brain edema, and glioma.

15. The method of claim 1, wherein the neurological disease or disorder involving an NKCC is stroke.

16. The method of claim 1, wherein the patient is a human.

17. A method of treating an ischemic event in a patient, comprising administering to the patient a therapeutically effective amount of a composition comprising: 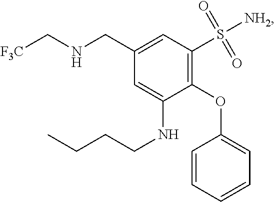 [SMILES: CCCCNc1cc(CNCC(F)(F)F)cc(S(N)(=O)=O)c1Oc1ccccc1]
or a pharmaceutically acceptable salt or solvate thereof.

18. The method of claim 17, wherein the composition is administered to the patient within 3 hours of the ischemic event.

19. The method of claim 17, wherein the ischemic event is a transient ischemic event.

20. A method of treating a stroke in a patient, comprising administering to the patient a composition comprising: 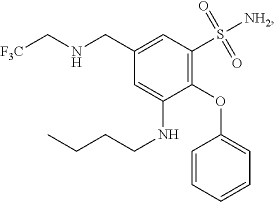 [SMILES: CCCCNc1cc(CNCC(F)(F)F)cc(S(N)(=O)=O)c1Oc1ccccc1]
or a pharmaceutically acceptable salt or solvate thereof, in an amount effective to ameliorate one or more effects of the stroke in the patient.
